(19) United States
(12) Patent Application Publication
(10) Pub. No.: US 2025/0099351 A1
(43) Pub. Date: Mar. 27, 2025

(54) HAIR TREATMENT COMPOSITIONS AND METHODS FOR SMOOTHING HAIR

(71) Applicant: L'OREAL, Paris (FR)

(72) Inventors: Vibha SHAH, Branchburg, NJ (US); Heather LEE, Wayne, NJ (US); Lisa Chuyin YE-TSE, Brooklyn, NY (US); Cho-Cho KHINE, Scotch Plains, NJ (US)

(21) Appl. No.: 18/355,442

(22) Filed: Jul. 20, 2023

(51) Int. Cl. A61K 8/365; A45D 7/04; A61K 8/73; A61K 8/898; A61Q 5/06; A61Q 5/12

(52) U.S. Cl. CPC A61K 8/365; A45D 7/04; A61K 8/738; A61K 8/898; A61Q 5/06; A61Q 5/12

(57) ABSTRACT

The instant disclosure is drawn to a hair treatment composition comprising: (a) citric acid, a salt thereof, or a combination thereof; (b) cyclodextrin, a salt thereof, or derivative thereof; (c) one or more cationic surfactants; (d) one or more nonionic surfactants or emulsifiers; (e) one or more fatty alcohols; (f) one more amino-functionalized silicones; and water. The hair treatment composition optionally includes one or more water-soluble solvents, non-silicone fatty compounds, and/or miscellaneous ingredients. The composition is particularly useful in methods for improving hair fiber alignment, reducing frizz, and imparting smoothness to the hair.

### FIELD OF THE DISCLOSURE

The present disclosure relates to hair treatment compositions and methods of treating hair with the compositions. Treatment with the compositions provide hair with improved fiber alignment, frizz control, and smoothness.

### BACKGROUND

Many consumers use cosmetic and care compositions to enhance the appearance of hair, e.g., by changing the color, style, or shape of the hair and/or by imparting various cosmetic properties to hair, such as shine and conditioning. Hair can become dry or damaged for various reasons, e.g., weather exposure, poor nutrition, mechanical treatments (e.g., brushing hair), styling treatments using chemicals, dying, heat, nutrition, etc. Even cleansing products can remove hair's natural oils causing dryness, which can lead to a dull appearance, split ends, and frizz.

Chemical treatments for hair include bleaching and coloring treatments to change the color the hair. Chemical treatments also include processes to permanently change the shape and structure of the hair, for example by perming, waving, relaxing or straightening the hair. These chemical treatments change the look of hair by changing its physical structure, which inevitably causes a certain degree of damage to the hair. Environmental factors, such as salt water, sunlight, and heat, are also known to damage hair. Damaged hair is characterized by unnatural changes to the protein structure of the individual hair strands or shafts.

The popularity and usage of oils for hair treatments has increased due to their effectiveness and simplicity. Commonly used oils include olive oil, mineral oil, avocado oil, apricot kernel oil, rice bran oil, and coconut oil. However, these treatments can leave the hair feeling greasy. In addition, the effects are not usually seen after more than several hours (e.g., 8 hours) of treatment and several treatments are usually required, making it time consuming and labor intensive.

Damage to hair results in split ends, dryness, hair that is easily broken, and hair that becomes “frizzy” and unmanageable. Because the visible portion of hair is dead, it has no ability to regenerate itself. There are numerous over the counter and salon treatments that purport to repair damaged hair. These include conditioners, hot oil treatments, hydrolyzed proteins, vitamin formulations, and exotic fruit, leaf, or root extracts. These treatments, however, provide only limited improvement to the hair. Therefore, hair treatment technologies that can straighten, relax, or style the hair without chemically damaging the hair are desired.

There is still a need for providing improved manageability of hair, for example, improved hair alignment, reduced unwanted volume (especially reduced frizz), and increased shine.

### SUMMARY OF THE DISCLOSURE

The instant disclosure is drawn to hair treatment compositions and methods for improving the look, feel, and style of hair. In particular, the hair treatment compositions and methods improve fiber alignment, reduce frizz, and impart smoothness. The treated hair is soft, shiny, conditioned, and has a healthy appearance. The compositions include a unique combination of a components that form a smooth coating on the surface of the hair. The smooth coating is surprisingly durable. Accordingly, the improved cosmetic properties imparted to the hair are long-lasting, resistant to humidity, and maintained even washing the hair.

The hair treatment compositions of the instant disclosure typically include:
(a) citric acid, a salt thereof, or a combination thereof;
(b) cyclodextrin or derivative thereof;
(c) one or more cationic surfactants;
(d) optionally, one or more nonionic surfactants or emulsifiers;
(e) one or more fatty alcohols;
(f) one or more amino-functionalized silicones;
(g) optionally, one or more water-soluble solvents;
(h) water.

Citric acid and its salts provide a myriad of benefits to hair. For example, it acts as an antioxidant, removes build-up and debris from the hair, and improves blood circulation in the scalp, which should nourish hair follicles and promotes growth. Its acidic pH is also useful for balancing the pH of the scalp, as many hair care products make it more alkaline. The citric acid in the instant compositions also interacts with cyclodextrin in a unique manner and improves film formation on the hair, which is further potentiated with heat. Sodium citrate (or trisodium citrate) is an example of a salt of citric acid.

Cyclodextrins are a family of cyclic oligosaccharides, comprised of a macrocyclic ring of glucose subunits joined by α-1,4 glycosidic bonds. Cyclodextrins are produced from starch by enzymatic conversion. Typical cyclodextrins contain a number of glucose monomers ranging from six to eight units in a ring, creating a cone shape, for example, α (alpha)-cyclodextrin gas 6 glucose subunits, β (beta)-cyclodextrin has 7 glucose subunits, and γ (gamma)-cyclodextrin has 8 glucose subunits. Nonlimiting examples of cyclodextrins for use in the compositions of the present disclosure include α-cyclodextrin, ß-cyclodextrin, γ-cyclodextrin, methyl-α-cyclodextrin, methyl-ß-cyclodextrin, methyl-γ-cyclodextrin, and mixtures thereof.

Nonlimiting examples of cationic surfactants include cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, stearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, and a mixture thereof.

The compositions typically include one or more nonionic surfactants or emulsifiers. Nonlimiting examples include nonionic surfactants or emulsifiers selected from alkoxylated fatty alcohols, fatty acid esters of polyoxyethylene glycol, ethoxylated mono or diglycerides, sorbitan esters, ethoxylated sorbitan esters, fatty acid glycol esters, ethylene oxide, alkyl(ether)phosphates, alkylpolyglucosides, and mixtures thereof. In various embodiments, at least one of the one or more nonionic surfactants or emulsifiers are alkoxylated, preferably ethoxylated. Fatty alcohol ethoxylates are particularly useful, for example, polyethylene glycol ethers of tridecyl alcohol (or other fatty alcohol).

Suitable fatty alcohols, if present, include but are not limited to those having a fatty group with a carbon chain of greater than 8 carbon atoms, 8 to 50 carbon atoms, 8 to 40 carbon atoms, 8 to 30 carbon atoms, 8 to 22 carbon atoms, 12 to 22 carbon atoms, or 12 to 18 carbon atoms. In some instances, the fatty group of the fatty alcohols has a carbon chain of 10 to 20 carbon atoms or 10 to 18 carbon atoms. Nonlimiting examples of fatty alcohols include decyl alcohol, undecyl alcohol, dodecyl alcohol, myristyl alcohol, lauryl alcohol, cetyl alcohol, stearyl alcohol, cetearyl alcohol (cetyl alcohol and stearyl alcohol), isostearyl alcohol, isocetyl alcohol, behenyl alcohol, linalool, oleyl alcohol, cis-4-t-butylcyclohexanol, isotridecyl alcohol, myricyl alcohol, and a mixture thereof.

Nonlimiting examples of amino-functionalized silicones include amodimethicone, bis-hydroxy/methoxy amodimethicone, bis-cetearyl amodimethicone, bis(C13-15 alkoxy) PG amodimethicone, aminopropyl phenyl trimethicone, aminopropyl dimethicone, bis-amino PEG/PPG-41/3 aminoethyl PG-propyl dimethicone, or a mixture thereof. In various embodiments, amodimethicone is preferred.

The compositions optionally include one or more water soluble solvents (also referred to as “water-soluble organic solvents”). Nonlimiting examples of water soluble solvents include glycerin, C₁-C₆ mono-alcohols, polyols (polyhydric alcohols), glycols, or a mixture thereof. In various embodiments, at least one of the one or more water soluble solvents is glycerin, a glycol (e.g., ethylene glycol, propylene glycol, butylene glycol, pentylene glycol, caprylyl glycol, etc.) or a combination thereof.

The compositions optionally include one or more non-silicone-based fatty compounds. Nonlimiting examples include oils, waxes, linear or branched alkanes, fatty esters, esters of fatty acids, esters of fatty alcohols, cetyl esters, triglycerides, or a mixture thereof. In various embodiments, the compositions include one or more linear or branched alkanes. In various embodiments, the compositions include an oil.

In various embodiments, the compositions include one or more miscellaneous ingredients. Nonlimiting examples of miscellaneous ingredients include preservatives, fragrances, pH adjusters, salts, chelating agents, buffers, antioxidants, flavonoids, vitamins, amino acids, botanical extracts, UV filtering agents, peptides, proteins, protein hydrolysates, and/or isolates, fillers (e.g., organic and/or inorganic fillers such as talc, calcium carbonate, silica, particular materials, etc.), emollients, composition colorants, or a mixture thereof.

The compositions are useful for improving the look, feel, and style of hair. Accordingly, the instant disclosure is drawn to methods for treating hair, for example, methods for improving the look, feel, or style of the hair, especially curly hair. More specifically, the instant disclosure is drawn to methods for improving fiber alignment, reducing frizz, and imparting smoothness to hair. Such methods typically include application of the composition to the hair. After allowing the composition to remain on the hair for a period of time (e.g., about 1 to about 10 minutes), the composition is rinsed from the hair. In various embodiments, the temperature of the hot iron is at least 100° C., for example, about 140° C. to about 190° C. The treated hair is soft, shiny, conditioned, with a healthy appearance. The improved cosmetic properties imparted to the hair are long-lasting and are maintained even after washing the hair.

In various embodiments, the compositions of the instant disclosure can be used in a routine with a heat treatment, for examples, treatment with a hot iron such as a flat iron. The compositions are applied to the hair, for example, after the hair has been washed, and allowed to remain on the hair for a period. The hair is subsequently rinsed, dried, and treated with a hot iron, preferably a flat iron. The treated hair is straightened with a high degree of fiber alignment, lacks frizz, and has a natural and healthy appearance.

### BRIEF DESCRIPTION OF THE DRAWINGS

Implementation of the present technology is described, by way of example only, with reference to the attached figure, wherein:

FIG. 1 shows hair swatches treated with a placebo, a composition of the instant disclosure, and comparative compositions, immediately after treatment.

FIG. 2 shows hair swatches treated with a placebo, a composition of the instant disclosure, and comparative compositions, after 1 hour in a humidity chamber (80%, 25° C.).

FIG. 3 shows hair swatches treated with a placebo, a composition of the instant disclosure, and comparative compositions, after being in a humidity chamber for 1 hour (80%, 25° C.) and subsequently washed.

FIG. 4 shows hair swatches treated with a placebo, a composition of the instant disclosure, and comparative compositions, after 1 hour in a humidity chamber (80%, 25° C.), subsequently washed, and again in the humidity chamber (80%, 25° C.) for 1 hour.

The various aspects are not limited to the arrangements, compositions, and instrumentality shown in the drawings.

### DETAILED DESCRIPTION OF THE DISCLOSURE

The instant disclosure is drawn to hair treatment compositions and methods for treating hair. The compositions include a unique combination of citric acid, cyclodextrin, and an amino-functionalized silicone. These components interact with one another in a synergistic-like manner. Treatment with the compositions improve fiber alignment, reduce frizz, and impart smoothness, to a surprising degree. The longevity of the improved fiber alignment, reduced frizz, and smoothness is also significant and surprising. Furthermore, hair treated with the compositions maintain these desirable cosmetic properties, even after washing. Thus, the benefits provided by the compositions are therefore long-lasting.

The hair treatment composition typically include:
(a) about 1 to about 8 wt. % of citric acid;
(b) about 0.5 to about 5 wt. % of a cyclodextrin or derivative thereof;
wherein a combined total amount of the citric acid of (a) and the cyclodextrin of (b) is about 2 to about 12 wt. %;
(c) about 1 to about 10 wt. % of one or more cationic surfactants;
(d) optionally, about 0.1 to about 5 wt. % of one or more nonionic surfactants or emulsifiers;
(e) about 1 to about 15 wt. % of one or more fatty alcohols;
(f) about 2 to about 18 wt. % of one or more amino-functionalized silicones;
(g) optionally, one or more water-soluble solvents;
(h) about 60 to about 85 wt. % of water;
   wherein all weight percentages are based on a total weight of the composition.

The pH of the composition can vary. Nonetheless, in various embodiments, a pH less than 7 (an acidic pH) is desirable. For example, the pH can be from about 3 to about 6.5, about 3 to about 6, about 3 to about 5.5, about 3 to about 5, or about 3 to about 4.5.

### (a) Citric Acid and Salts Thereof

The total amount of citric acid, salts thereof, or combination thereof will vary. Nonetheless, in various embodiments, the hair treatment composition includes about 1 to about 5 wt. % of citric acid, salts thereof, or a combination thereof, based on a total weight of the composition. In further embodiments, the hair treatment composition includes about 1 to about 4 wt. %, about 1 to about 3 wt. %, about 2 to about 5 wt. %, about 2 to about 4 wt. %, about 2 to about 3 wt. %, about 2.5 to about 5 wt. %, about 2.5 to about 4 wt. %, about 2.5 to about 3 wt. %, or about 1, about 1.5, about 2, about 2.5, about 3, about 3.5, about 4, about 4.5, or about 5 wt. % of citric acid, salts thereof, or a combination thereof, based on a total weight of the composition.

### (b) Cyclodextrin and Derivatives

Compositions according to the disclosure comprise at least one cyclodextrin or derivative thereof. As used herein, the term “cyclodextrins” includes slats of carboxylic acid, whether or not expressly stated. Cyclodextrins are a family of cyclic oligosaccharides consisting of a macrocyclic ring of glucose subunits joined by α-1,4 glycosidic bonds.

The cyclodextrins that can be used include those of the formula:

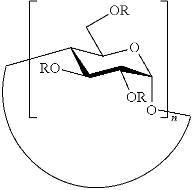

wherein:
R is chosen from H, CH₃, or a hydroxypropyl group, and
n ranges from 6-8.

For example, in embodiments where R═H, the cyclodextrin may be α-cyclodextrin (n=6), ß-cyclodextrin (n=7), or γ-cyclodextrin (n=8). By way of example, α-cyclodextrin sold by the company WACKER under the name CAVAMAX WB1 PHARMA, ß-cyclodextrin sold by the company WACKER under the name CAVAMAX W7 PHARMA, or γ-cyclodextrin sold by the company WACKER under the name CAVAMAX W8 PHARMA can be used.

In other embodiments where R═CH₃, the cyclodextrin may be a methyl-cyclodextrin, such as methyl-α-cyclodextrin (n=6), methyl-ß-cyclodextrin (n=7), or methyl-γ-cyclodextrin (n=8). For example, the methyl-ß-cyclodextrin sold by the company WACKER under the name CAVASOL W7 may be chosen.

In various embodiments, the at least one cyclodextrin may comprise a mixture of cyclodextrins and/or derivatives thereof. For example, the at least one cyclodexctrin may be a mixture of α-cyclodextrin, ß-cyclodextrin, and/or γ-cyclodextrin. In another embodiment, the at least one cyclodextrin includes ß-cyclodextrin. In yet a further embodiment, the cyclodextrin is only ß-cyclodextrin, and no other cyclodextrins or derivatives thereof are present in the composition.

In one embodiment, the compositions according to the present disclosure includes ß-cyclodextrin in an amount ranging from about 0.1% to about 10%, such as from 0.2% to about 8%, from about 0.3% to about 7%, from about 0.4% to about 6%, from about 1% to about 10%, from about 1% to about 8%, from about 1% to about 5%, from about 1% to about 3% by weight, relative to the total weight of the hair treatment composition.

The total amount of cyclodextrin in the hair treatment compositions will vary. Nonetheless, in various embodiments, the hair treatment composition includes about 0.5 to about 5 wt. % of cyclodextrin, based on a total weight of the composition. In further embodiments, the hair treatment composition includes 0.5 to about 4 wt. %, about 0.5 to about 3 wt. %, about 0.5 to about 2 wt. %, about 1 to about 5 wt. %, about 1 to about 4 wt. %, about 1 to about 3 wt. %, about 1 to about 2 wt. %, about 1.5 to about 5 wt. %, about 1.5 to about 4 wt. %, about 1.5 to about 3 wt. %, about 1.5 to about 2 wt. %, about 0.5 wt. %, about 1 wt. %, about 1.25 wt. %, about 1.5 wt. %, about 2 wt. %, about 2.5 wt. %, about 3 wt. %, about 3.5 wt. %, about 4 wt. %, about 4.5 wt. %, or about 5 wt. %, based on a total weight of the composition.

### Combination of Citric Acid/Salts and Cyclodextrin

The total combined amount of the citric acid, salts thereof, or combination of (a) and the cyclodextrin or derivatives of (b) will vary. Nonetheless, in various embodiments the total combined amount of the citric acid, salts thereof, or combination of (a) and the cyclodextrin and derivatives of (b) is about 1 to about 12 wt. %, based on a total weight of the hair treatment composition. In further embodiments, the total combined amount of the citric acid, salts thereof, or combination of (a) and the cyclodextrin and derivatives of (b) is about 1 to about 10 wt. %, about 1 to about 8 wt. %, about 1 to about 6 wt. %, about 1 to about 5 wt. %, about 2 to about 12 wt. %, about 2 to about 10 wt. %, about 2 to about 8 wt. %, about 2 to about 6 wt. %, about 2 to about 5 wt. %, about 3 to about 12 wt. %, about 3 to about 10 wt. %, about 3 to about 8 wt. %, about 3 to about 6 wt. %, about 3 to about 5 wt. %, or about 1 wt. %, 2 wt. %, 3 wt. %, 4 wt. %, 5 wt. %, 6 wt. %, 7 wt. %, or 8 wt. %, based on a total weight of the hair treatment composition.

The weight ratio of the citric acid, salts thereof, or combination of (a) to the cyclodextrin or derivatives of (b) will vary. Nonetheless, in various embodiments, the citric acid, salts thereof, or combination of (a) and the cyclodextrin or derivatives of (b) are in a weight ratio of about of about 8:1 to about 1:2 ((a):(b)). In further embodiments, the citric acid, salts thereof, or combination of (a) and the cyclodextrin or derivatives of (b) are in a weight ratio of 6:1 to about 1:2, about 5:2 to about 1:2, about 4:1 to about 1:2, about 3:1 to about 1:2, about 2:1 to about 1:2, about 8:1 to about 1:1, about 6:1 to about 1:1, about 5:1 to about 1:1, about 4:1 to about 1:1, about 3:1 to about 1:1, about 2:1 to about 1:1, about 1.2:1, about 1.3:1, about 1.4:1, about 1.5:1, about 1.6:1, or about 1.8:1 ((a):(b)).

The mole ratio of the citric acid, salts thereof, or combination of (a) to the cyclodextrin or derivatives of (b) will vary. Nonetheless, in various embodiments, the citric acid, salts thereof, or combination of (a) and the cyclodextrin or derivatives of (b) are in a mole ratio of about 20:1 to about 3:1. In further embodiments, the citric acid, salts thereof, or combination of (a) and the cyclodextrin or derivatives of (b) are in a mole ratio of about 18:1 to about 3:1, about 15:1 to about 3:1, about 20:1 to about 5:1, about 18:1 to about 5:1, about 15:1 to about 5:1, about 20:1 to about 8:1, about 18:1 to about 8:1, about 15:1 to about 8:1, about 20:1 to about 10:1, about 18:1 to about 10:1, about 15:1 to about 10:1, about 14:1, about 13:1, about 12:1, or about 11:1.

In various embodiments, the citric acid, salts thereof, or combination of (a) and the cyclodextrin of (b) are combined with one another before being added into the hair treatment compositions of the instant disclosure. For example, the cyclodextrin is preferably solubilized in the citric acid to form a solubilized combination of citric acid and cyclodextrin. The combination can be heated to facilitate or hasten the dissolution of the cyclodextrin. The solubility of cyclodextrin in water is not always ideal. Therefore, combining the cyclodextrin with the citric acid and dissolving the cyclodextrin in citric acid before adding combining the combination with other components of the hair treatment composition can be beneficial.

### (c) Cationic Surfactants

The term “cationic surfactant” as defined by the instant disclosure is a surfactant that may be positively charged when it is contained in the hair treatment compositions according to the disclosure. The cationic surfactant may bear one or more positive permanent charges or may contain one or more functional groups that are cationizable in the composition according to the disclosure.

Mono-alkyl cationic surfactants useful herein are primary, secondary, and tertiary amines having one long alkyl or alkenyl group of from about 12 to about 30 carbon atoms, preferably from 16 to 24 carbon atoms, more preferably from 18 to 22 alkyl group. For example, mono-alkyl cationic surfactants include mono-alkyl trimonium halide compounds. Nonlimiting examples of mono-alkyl trimonium halide compounds include cetrimonium chloride, steartrimonium chloride, behentrimonium chloride, cocotrimonium chloride, cocamidopropyltrimonium chloride. Preferred are cetrimonium chloride, steartrimonium chloride and behentrimonium chloride.

In various embodiments, the hair treatment compositions includes behentrimonium chloride, cetrimonium chloride, or a combination thereof.

Mono-alkyl cationic surfactants also include mono-alkyl amidoamines. Particularly useful are tertiary amidoamines having an alkyl group of from about 12 to about 22 carbon atoms, preferably from about 16 to about 22 carbon atoms. Exemplary tertiary amido amines include: stearamidopropyldimethylamine, stearamidopropyl-diethylamine, stearamidoethyldiethylamine, stearamidoethyldimethylamine, palmitamidopropyldimethylamine, palmitamidopropyldiethylamine, palmitamidoethyl-diethylamine, palmitamidoethyldimethylamine, behenamidopropyldimethylamine, behenamidopropyldiethylamine, behenamidoethyldiethylamine, behenamidoethyl-dimethylamine, arachidamidopropyldimethylamine, arachidamidopropyldiethylamine, arachidamidoethyldiethylamine, arachidamidoethyldimethylamine, diethylaminoethyl-stearamide, and a combination thereof.

Di-alkyl cationic surfactants includes those of formula (I) and salts thereof:

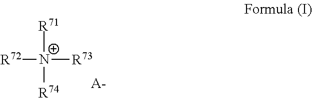

wherein two of R⁷¹, R⁷², R⁷³, and R⁷⁴ are selected from an aliphatic group of from 12 to 30 carbon atoms, preferably from 16 to 24 carbon atoms, more preferably from 18 to 22 carbon atoms or an aromatic, alkoxy, polyoxyalkylene, alkylamido, hydroxyalkyl, aryl or alkylaryl group having up to about 30 carbon atoms;
the remainder of R⁷¹, R⁷², R⁷³ and R⁷⁴ are independently selected from an aliphatic group of from 1 to about 8 carbon atoms, preferably from 1 to 3 carbon atoms or an aromatic, alkoxy, polyoxyalkylene, alkylamido, hydroxyalkyl, aryl or alkylaryl group having up to about 8 carbon atoms; and
A⁻ is an anion, for example, a halide, such as chloride or bromide, a C1-C4 alkyl sulfate such as methosulfate and ethosulfate, and mixtures thereof.

The aliphatic groups for Formula (I) can contain, in addition to carbon and hydrogen atoms, ether linkages, and other groups such as amino groups. The longer chain aliphatic groups, e.g., those of about 16 carbons, or higher, can be saturated or unsaturated. Preferably, two of R⁷¹, R⁷², R⁷³ and R⁷⁴ are selected from an alkyl group of from 12 to 30 carbon atoms, preferably from 16 to 24 carbon atoms, more preferably from 18 to 22 carbon atoms; and the remainder of R⁷¹, R⁷², R⁷³ and R⁷⁴ are independently selected from CH₃, C₂H₅, C₂H₄OH, CH₂C₆H₅, and mixtures thereof.

Nonlimiting examples of di-alkyl cationic surfactants of Formula (I) include dialkyl (14-18) dimethyl ammonium chloride, ditallow alkyl dimethyl ammonium chloride, dihydrogenated tallow alkyl dimethyl ammonium chloride, distearyl dimethyl ammonium chloride, dicetyl dimethyl ammonium chloride, dicetyldimonium chloride, dicetyldimonium bromide, and a combination thereof.

In various embodiments, the one or more di-alkyl cationic surfactants are selected from di-alkyl dimonium halide compounds. Nonlimiting examples include dialkyl (14-18) dimethyl ammonium chloride, ditallow alkyl dimethyl ammonium chloride, dihydrogenated tallow alkyl dimethyl ammonium chloride, distearyl dimethyl ammonium chloride, dicetyl dimethyl ammonium chloride, dicetyldimonium chloride, dicetyldimonium bromide, and a combination thereof. In a preferred embodiment, the di-alkyl dimonium halide compounds are selected from dicetyldimonium chloride, dicetyldimonium bromide, and a combination thereof. In a preferred embodiment, the hair treatment composition includes dicetyldimonium chloride.

The total amount of the one or more cationic surfactants in the hair treatment composition will vary. Nonetheless, in various embodiments, the hair treatment compositions include about 1 to about 10 wt. % of the one or more cationic surfactants, based on a total weight of the composition. In further embodiments, the hair treatment composition includes about 1 to about 8 wt. %, about 1 to about 6 wt. %, about 1 to about 5 wt. %, about 1 to about 4 wt. %, about 1 to about 3 wt. %, about 1.5 to about 10 wt. %, 1.5 to about 8 wt. %, about 1.5 to about 6 wt. %, about 1.5 to about 5 wt. %, about 1.5 to about 4 wt. %, about 1.5 to about 3 wt. %, about 2 to about 10 wt. %, about 2 to about 8 wt. %, about 2 to about 6 wt. %, about 2 to about 5 wt. %, about 2 to about 4 wt. %, about 2 to about 3 wt. %, or about 1 wt. %, 1.5 wt. %, 2 wt. %, about 2.5 wt. %, about 3 wt. %, about 3.5 wt. %, about 4 wt. %, about 4.5 wt. %, about 5 wt. %, about 6 wt. %, about 8 wt. %, or about 10 wt. % of the one or more cationic surfactants.

### (d) Nonionic Surfactants or Emulsifiers

The terms “nonionic surfactant” and “nonionic emulsifier” are used interchangeably in the instant disclosure and therefore can be referred to as “nonionic emulsifying surfactants.” The nonionic surfactant or emulsifier may have an HLB (hydrophilic-lipophilic balance) ranging from 1 to 7.9 or greater than or equal to 8. “HLB” refers to the “hydrophilic-lipophilic balance” associated with nonionic surfactants or emulsifiers. In particular, “HLB” value relates to the ratio of hydrophilic groups and lipophilic groups in emulsifiers, and also relates to solubility of the emulsifiers. Lower HLB emulsifiers (such as those with HLB values ranging from 1 to 7.9) are more soluble in oils (lipophilic material) and are more appropriate for use in water-in-oil (W/O) emulsions. Higher HLB emulsifiers (such as those with HLB values higher than 8) are more soluble in water (hydrophilic material) and are more appropriate for oil-in-water (O/W) emulsions.

Nonlimiting examples of nonionic surfactants or emulsifiers include alkyl and polyalkyl esters of poly(ethylene oxide), alkyl and polyalkyl ethers of poly(ethylene oxide), optionally polyoxyethylenated alkyl and polyalkyl esters of sorbitan, optionally polyoxyethylenated alkyl and polyalkyl ethers of sorbitan, alkyl and polyalkyl glycosides or polyglycosides, in particular alkyl and polyalkyl glucosides or polyglucosides, alkyl and polyalkyl esters of sucrose, optionally polyoxyethylenated alkyl and polyalkyl esters of glycerol, and optionally polyoxyethylenated alkyl and polyalkyl ethers of glycerol, and mixtures thereof. Preferably, the non-ionic surfactant(s) may be chosen from alkyl and polyalkyl esters of poly(ethylene oxide), alkyl and polyalkyl ethers of poly(ethylene oxide), optionally polyoxyethylenated alkyl and polyalkyl esters of sorbitan, optionally polyoxyethylenated alkyl and polyalkyl ethers of sorbitan, optionally polyoxyethylenated alkyl and polyalkyl esters of glycerol, and optionally polyoxyethylenated alkyl and polyalkyl ethers of glycerol, and mixtures thereof.

(1) Alkyl and polyalkyl esters of poly(ethylene oxide) that are preferably used are those containing at least one C8-C30 alkyl radical, with a number of ethylene oxide (EO) units ranging from 2 to 200. Mention may be made, for example, of (INCI name) PEG-20 stearate, PEG-40 stearate, PEG-100 stearate, PEG-20 laurate, PEG-8 laurate, PEG-40 laurate, PEG-150 distearate, PEG-7 cocoate, PEG-9 cococate, PEG-8 oleate, PEG-10 oleate and PEG-40 hydrogenated castor oil.

(2) Alkyl and polyalkyl ethers of poly(ethylene oxide) that are preferably used are those containing at least one C8-C30 alkyl radical, with a number of ethylene oxide (EO) units ranging from 3 to 200. Mention may be made, for example, of laureth-3, laureth-4, laureth-7, laureth-23, ceteth-5, ceteth-7, ceteth-15, ceteth-23, oleth-5, oleth-7, oleth-10, oleth-12, oleth-20, oleth-50, phytosterol 30 EO, steareth-6, steareth-20, steareth-21, steareth-40, steareth-100, beheneth 100, ceteareth-7, ceteareth-10, ceteareth-15, ceteareth-25, pareth-3, pareth-23, C12-15 pareth-3, C12-13 pareth-4, C12-13 pareth-23, trideceth-3, trideceth-4, trideceth-5, trideceth-6, trideceth-7 and trideceth-10, and mixtures thereof.

(3) Polyoxyethylenated alkyl and polyalkyl esters of sorbitan that are preferably used are those with a number of ethylene oxide (EO) units ranging from 0 to 100. Mention may be made, for example, of sorbitan laurate, sorbitan laurate 4 EO, sorbitan laurate 20 EO (polysorbate 20), sorbitan palmitate 20 EO (polysorbate 40), sorbitan stearate 20 EO (polysorbate 60), sorbitan oleate 20 EO (polysorbate 80) and sorbitan trioleate 20 EO (polysorbate 85).

(4) Polyoxyethylenated alkyl and polyalkyl ethers of sorbitan that are preferably used are those with a number of ethylene oxide (EO) units ranging from 0 to 100.

The compositions of the instant disclosure may include one or more alkanolamides. Non-limiting examples alkanolamides include fatty acid alkanolamides. The fatty acid alkanolamides may be fatty acid monoalkanolamides or fatty acid dialkanolamides or fatty acid isoalkanolamides, and may have a C₂₋₈ hydroxyalkyl group (the C₂₋₈ chain can be substituted with one or more than one —OH group). Non-limiting examples include fatty acid diethanolamides (DEA) or fatty acid monoethanolamides (MEA), fatty acid monoisopropanolamides (MIPA), fatty acid diisopropanolamides (DIPA), and fatty acid glucamides (acyl glucamides).

Suitable fatty acid alkanolamides include those formed by reacting an alkanolamine and a C6-C36 fatty acid. Examples include, but are not limited to: oleic acid diethanolamide, myristic acid monoethanolamide, soya fatty acids diethanolamide, stearic acid ethanolamide, oleic acid monoisopropanolamide, linoleic acid diethanolamide, stearic acid monoethanolamide (Stearamide MEA), behenic acid monoethanolamide, isostearic acid monoisopropanolamide (isostearamide MIPA), erucic acid diethanolamide, ricinoleic acid monoethanolamide, coconut fatty acid monoisopropanolamide (cocoamide MIPA), coconut acid monoethanolamide (Cocamide MEA), palm kernel fatty acid diethanolamide, coconut fatty acid diethanolamide, lauric diethanolamide, polyoxyethylene coconut fatty acid monoethanolamide, coconut fatty acid monoethanolamide, lauric monoethanolamide, lauric acid monoisopropanolamide (lauramide MIPA), myristic acid monoisopropanolamide (Myristamide MIPA), coconut fatty acid diisopropanolamide (cocamide DIPA), and mixtures thereof.

In some instances, the fatty acid alkanolamides preferably include cocamide MIPA, cocamide DEA, cocamide MEA, cocamide DIPA, and mixtures thereof. In particular, the fatty acid alkanolamide may be cocamide MIPA, which is commercially available under the tradename EMPILAN from Innospec Active Chemicals.

Fatty acid alkanolamides include those of the following structure:

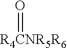

wherein R₄ is an alkyl chain of 4 to 20 carbon atoms (R₄ may be, for example, selected from lauric acid, coconut acid, palmitic acid, myristic acid, behenic acid, babassu fatty acid, isostearic acid, stearic acid, corn fatty acid, soy fatty acid, shea butter fatty acids, caprylic acid, capric acid, and mixtures thereof);
R₆ is selected from —CH₂OH, —CH₂CH₂OH, —CH₂CH₂CH₂OH, —CH₂(CHOH)₄CH₂OH, -benzyl, and mixtures thereof;
R₆ is selected from —H, —CH₃, —CH₂OH, —CH₂CH₃, —CH₂CH₂OH, —CH₂CH₂CH₂OH, —CH₂(CHOH)₄CH₂OH, -benzyl, and mixtures thereof.

In some instances, the one or more of the fatty acid alkanolamides include one or more acyl glucamides, for example, acyl glucamides having a carbon chain length of 8 to 20. Non-limiting examples include lauroyl/myristoyl methyl glucamide, capryloyl/capryl methyl glucamide, lauroyl methyl glucamide, myristoyl methyl glucamide, capryloyl methyl glucamide, capryl methyl glucamide, cocoyl methyl glucamide, capryloyl/caproyl methyl glucamide, cocoyl methyl glucamide, lauryl methylglucamide, oleoyl methylglucamide oleate, stearoyl methylglucamide stearate, sunfloweroyl methylglucamide, and tocopheryl succinate methylglucamide.

The compositions of the instant disclosure may include one or more alkyl polyglucosides. Non-limiting examples of alkyl polyglucosides include those having the following formula:

R¹—O—(R²O)ₙ—Z(x)
wherein R¹ is an alkyl group having 8-18 carbon atoms;
R² is an ethylene or propylene group;
Z is a saccharide group with 5 to 6 carbon atoms;
n is an integer from 0 to 10; and
x is an integer from 1 to 5.

Useful alkyl poly glucosides include lauryl glucoside, octyl glucoside, decyl glucoside, coco glucoside, caprylyl/capryl glucoside, and sodium lauryl glucose carboxylate. Typically, the at least one alkyl poly glucoside compound is selected from the group consisting of lauryl glucoside, decyl glucoside and coco glucoside. In some instances, decyl glucoside is particularly preferred.

The compositions of the instant disclosure may include one or more miscellaneous nonionic surfactants or emulsifiers. Nonlimiting examples include alcohols, alpha-diols, alkylphenols and esters of fatty acids, being ethoxylated, propoxylated or glycerolated and having at least one fatty chain comprising, for example, from 8 to 18 carbon atoms, it being possible for the number of ethylene oxide or propylene oxide groups to range from 2 to 50, and for the number of glycerol groups to range from 1 to 30. Maltose derivatives may also be mentioned. Non-limiting mention may also be made of copolymers of ethylene oxide and/or of propylene oxide; condensates of ethylene oxide and/or of propylene oxide with fatty alcohols; polyethoxylated fatty amides comprising, for example, from 2 to 30 mol of ethylene oxide; polyglycerolated fatty amides comprising, for example, from 1.5 to 5 glycerol groups, such as from 1.5 to 4; ethoxylated fatty acid esters of sorbitan comprising from 2 to 30 mol of ethylene oxide; ethoxylated oils from plant origin; fatty acid esters of sucrose; fatty acid esters of polyethylene glycol; polyethoxylated fatty acid mono or diesters of glycerol (C₆-C₂₄)alkylpolyglycosides; N—(C₆-C₂₄)alkylglucamine derivatives, amine oxides such as (C₁₀-C₁₄)alkylamine oxides or N—(C₁₀-C₁₄)acylaminopropylmorpholine oxides; and mixtures thereof.

Such nonionic surfactants may preferably be chosen from polyoxyalkylenated or polyglycerolated nonionic surfactants. The oxyalkylene units are more particularly oxyethylene or oxypropylene units, or a combination thereof, and are preferably oxyethylene units.

In some cases, the nonionic surfactant may be selected from esters of polyols with fatty acids with a saturated or unsaturated chain containing for example from 8 to 24 carbon atoms, preferably 12 to 22 carbon atoms, and alkoxylated derivatives thereof, preferably with a number of alkyleneoxide of from 10 to 200, and more preferably from 10 to 100, such as glyceryl esters of a C₈-C₂₄, preferably C₁₂-C₂₂, fatty acid or acids and alkoxylated derivatives thereof, preferably with a number of alkyleneoxide of from 10 to 200, and more preferably from 10 to 100; polyethylene glycol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sorbitol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sugar (sucrose, glucose, alkylglycose) esters of a C₈-C₂₄, preferably C₁₂-C₂₂, fatty acid or acids and alkoxylated derivatives thereof, preferably with a number of alkyleneoxide of from 10 to 200, and more preferably from 10 to 100; ethers of fatty alcohols; ethers of sugar and a C₈-C₂₄, preferably C₁₂-C₂₂, fatty alcohol or alcohols; and mixtures thereof.

Examples of ethoxylated fatty esters that may be mentioned include the adducts of ethylene oxide with esters of lauric acid, palmitic acid, stearic acid or behenic acid, and mixtures thereof, especially those containing from 9 to 100 oxyethylene groups, such as PEG-9 to PEG-50 laurate (as the CTFA names: PEG-9 laurate to PEG-50 laurate); PEG-9 to PEG-50 palmitate (as the CTFA names: PEG-9 palmitate to PEG-50 palmitate); PEG-9 to PEG-50 stearate (as the CTFA names: PEG-9 stearate to PEG-50 stearate); PEG-9 to PEG-50 palmitostearate; PEG-9 to PEG-50 behenate (as the CTFA names: PEG-9 behenate to PEG-50 behenate); polyethylene glycol 100 EO monostearate (CTFA name: PEG-100 stearate); and mixtures thereof.

As glyceryl esters of fatty acids, glyceryl stearate (glyceryl mono-, di- and/or tristearate) (CTFA name: glyceryl stearate) or glyceryl ricinoleate and mixtures thereof can in particular be cited.

As glyceryl esters of C₈-C₂₄ alkoxylated fatty acids, polyethoxylated glyceryl stearate (glyceryl mono-, di- and/or tristearate) such as PEG-20 glyceryl stearate can for example be cited.

Mixtures of these surfactants, such as for example the product containing glyceryl stearate and PEG-100 stearate, marketed under the name ARLACEL 165 by Uniqema, and the product containing glyceryl stearate (glyceryl mono- and distearate) and potassium stearate marketed under the name TEG1 N by Goldschmidt (CTFA name: glyceryl stearate SE), can also be used.

The total amount of the one or more nonionic surfactants or emulsifiers in the hair treatment compositions will vary. Nonetheless, in various embodiments, the hair treatment compositions include about 0.1 to about 10 wt. % of the one or more nonionic surfactants or emulsifiers. In further embodiments, the compositions include about 0.1 to about 8 wt. %, about 0.1 to about 5 wt. %, about 0.1 to about 3 wt. %, about 0.5 to about 10 wt. %, about 0.5 to about 8 wt. %, about 0.5 to about 5 wt. %, about 0.5 to about 3 wt. %, about 1 to about 10 wt. %, about 1 to about 8 wt. %, about 1 to about 5 wt. %, about 1 to about 3 wt. %, about 0.1 wt. %, about 0.5 wt. %, about 1 wt. %, about 1.5 wt. % about 2 wt. %, about 2.5 wt. %, about 3 wt. % about 5 wt. %, about 6 wt. %, about 8 wt. %, or about 10 wt. % of the one or more nonionic surfactants or emulsifiers, based on a total weight of the compositions.

### (e) Fatty Alcohols

The term “fatty alcohol” means an alcohol comprising at least one hydroxyl group (OH), and comprising at least 8 carbon atoms, and which is neither oxyalkylenated (in particular neither oxyethylenated nor oxypropylenated) nor glycerolated. The fatty alcohols can be represented by: R—OH, wherein R denotes a saturated (alkyl) or unsaturated (alkenyl) group, linear or branched, comprising from 8 to 40 carbon atoms, preferably 10 to 30 carbon atoms, more preferably 12 to 24 carbon atoms, and even more preferably 14 to 22 carbon atoms.

The fatty alcohol(s) may be liquid or solid. In some instances, it is preferable that the cosmetic compositions include at least one solid fatty alcohol. The solid fatty alcohols that can be used include those that are solid at ambient temperature and at atmospheric pressure (25° C., 780 mmHg), and are insoluble in water, that is to say they have a water solubility of less than 1% by weight, preferably less than 0.5% by weight, at 25° C., 1 atm.

The solid fatty alcohols may be represented by: R—OH, wherein R denotes a linear alkyl group, optionally substituted with one or more hydroxyl groups, comprising from 8 to 40 carbon atoms, preferably 10 to 30 carbon atoms, more preferably 12 to 24 carbon atoms, and even more preferably 14 to 22 carbon atoms.

Non-limiting examples of useful fatty alcohols include lauryl alcohol or lauryl alcohol (1-dodecanol); myristic or myristyl alcohol (1-tetradecanol); cetyl alcohol (1-hexadecanol); stearyl alcohol (1-octadecanol); arachidyl alcohol (1-eicosanol); behenyl alcohol (1-docosanol); lignoceryl alcohol (1-tetracosanol); ceryl alcohol (1-hexacosanol); montanyl alcohol (1-octacosanol); myricylic alcohol (1-triacontanol), and mixtures thereof.

In certain embodiments, the one or more fatty alcohols have from 12 to 24 carbon atoms. Specific nonlimiting examples include cetyl alcohol, stearyl alcohol, cetearyl alcohol, behenyl alcohol, lauryl alcohol, myristic or myristyl alcohol, arachidyl alcohol, lignoceryl alcohol, or mixtures thereof.

Preferably, the cosmetic composition includes one or more solid fatty alcohols, for example, chosen from cetyl alcohol, stearyl alcohol, behenyl alcohol and mixtures thereof, preferably cetyl alcohol, behenyl alcohol, cetearyl alcohol, and mixtures thereof.

The liquid fatty alcohols, in particular those containing C₁₀-C₃₄, preferably have branched carbon chains and/or have one or more, preferably 1 to 3 double bonds. They are preferably branched and/or unsaturated (C=C double bond) and contain from 12 to 40 carbon atoms.

The liquid fatty alcohols may be represented by: R—OH, wherein R denotes a C₁₂-C₂₄ branched alkyl group or an alkenyl group (comprising at least one C₁₂-C₂₄ double bond C=C), R being optionally substituted by a or more hydroxy groups. Preferably, the liquid fatty alcohol is a branched saturated alcohol. Preferably, R does not contain a hydroxyl group. These include oleic alcohol, linoleic alcohol, linolenic alcohol, isocetyl alcohol, isostearyl alcohol, 2-octyl-1-dodecanol, 2-butyloctanol, 2-hexyl-1-decanol, 2-decyl-1-tetradecanol, 2-tetradecyl-1-cetanol and mixtures thereof. Preferably, the liquid fatty alcohol is 2-octyl-1-dodecanol.

In some instances, the cosmetic compositions include one or more fatty alcohols selected from decyl alcohol, undecyl alcohol, dodecyl, myristyl, cetyl alcohol, stearyl alcohol, cetearyl alcohol, isostearyl alcohol, isocetyl alcohol, behenyl alcohol, linalool, oleyl alcohol, myricyl alcohol and a mixture thereof. In some instances, the cosmetic compositions preferably include cetyl alcohol, behenyl alcohol, and cetearyl alcohol.

The total amount of the one or more fatty alcohols in the hair treatment composition will vary. Nonetheless, in various embodiments, the hair treatment composition includes about 1 to about 15 wt. % of the one or more fatty alcohols, based on a total weight of the composition. In further embodiments, the hair treatment composition includes about 2 to about 15 wt. %, about 3 to about 15 wt. %, about 4 to about 15 wt. %, about 5 to about 15 wt. %, about 6 to about 15 wt. %, about 1 to about 12 wt. %, about 2 to about 12 wt. %, about 3 to about 12 wt. %, about 4 to about 12 wt. %, about 5 to about 12 wt. %, about 6 to about 12 wt. %, about 1 to about 10 wt. %, about 2 to about 10 wt. %, about 3 to about 10 wt. %, about 4 to about 10 wt. %, about 5 to about 10 wt. %, about 6 to about 10 wt. %, about 1 to about 8 wt. %, about 2 to about 8 wt. %, about 3 to about 8 wt. %, about 4 to about 8 wt. %, about 5 to about 10 wt. %, about 6 to 8 wt. %, about 2 wt. %, about 3 wt. %, about 4 wt. %, about 5 wt. %, about 6 wt. %, about 7 wt. %, or about 8 wt. % of the one or more fatty alcohols.

### (f) Amino-Functionalized Silicones

The term “amino-functionalized silicone” or “amino silicones” means a silicone containing at least one primary amino, secondary amino, tertiary amino and/or quaternary ammonium group. The structure of the amino-functionalized silicone may be linear or branched, cyclic or non-cyclic. The amino functional group may be at any position in the silicone molecule, preferably at the end of the backbone (for example, in the case of amodimethicones) and/or in the side chain.

In some instances, an amino-functionalized silicones is selected from compounds having the following formula:

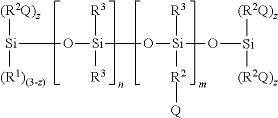

wherein each R¹ is independently selected from a C₁₋₃₀ alkyl group, a C₁₋₃₀ alkoxy 15 group, a C₅₋₃₀ aryl group, a C₆₋₃₀ aralkyl group, a C₆₋₃₀ aralkyloxy group, a C₁₋₃₀ alkaryl group, a C₁₋₃₀ alkoxyaryl group, and a hydroxy group (preferably, each R¹ is independently selected from a C₁₋₃₀ alkyl group, a C₁₋₃₀ alkoxy group and a hydroxy group);
each R² is independently a divalent alkylene radical having one to ten carbon 20 atoms (preferably, R² is a divalent alkylene radical having three to six carbon atoms);
each R³ is independently selected from a C₁₋₃₀ alkyl group, a C₅₋₃₀ aryl group, a C₆₋₃₀ aralkyl group and a C₁₋₃₀ alkaryl group (preferably, each R³ is independently selected from of a C₁₋₃₀ alkyl group);
Q is a monovalent radical selected from —NR⁴₂ and —NR⁴(CH₂)ₓNR⁴₂;
each R⁴ is independently selected from a hydrogen and a C₁₋₄ alkyl group;
x is 2 to 6;
z is 0 or 1;
n is 25 to 3,000 (preferably, 25 to 2,000; more preferably, 25 to 1,000; most preferably 25 to 500); and
m is 0 to 3,000 (preferably, 0 to 2,000; more preferably, 0 to 1,000; most preferably, 0 to 100);
with the proviso that at least 50 mol % of the total number of R¹ and R³ groups are methyl and with the proviso that when m is 0, z is 1.

Preferred R¹ groups include methyl, methoxy, ethyl, ethoxy, propyl, propoxy, isopropyl, isopropoxy, butyl, butoxy, isobutyl, isobutoxy, phenyl, xenyl, benzyl, phenylethyl, tolyl and hydoxy. Preferred R² divalent alkylene radicals include trimethylene, tetramethylene, pentamethylene, —CH₂CH(CH₃)CH₂ and CH₂CH₂CH(CH₃)CH₂.

Preferred R³ groups include methyl, ethyl, propyl, isopropyl, butyl, isobutyl, phenyl, xenyl, benzyl, phenylethyl and tolyl. Preferred R⁴ groups include methyl, ethyl, propyl, isopropyl, butyl and isobutyl. When z is 0, the amino-functionalized silicine has only pendant amine functional substituents in the polymer chain. When z is 1, the amino-functional silicone may have only terminal amine functional substituents (e.g., m=0) or may have both terminal and pendant amine functional substituents in the polymer chain (e.g., m>0). Preferably, n+m is 50 to 1,000. More preferably, n+m is 50 to 750. Still more preferably, n+m is 50 to 500. Most preferably, n+m is 50 to 250.

In some instances, the amino-functionalized silicones are alkoxylated and/or hydroxylated amino silicones. Suitable alkoxylated and/or hydroxylated amino silicones may be selected from compounds of the following formula:

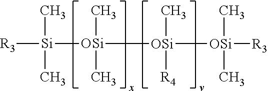

wherein R₃ is hydroxyl or OR₅, R₅ is a C₁ to C₄ alkyl group, R₄ is a group with structure according to the following formula:

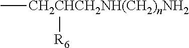

R₆ is a C₁ to C₄ alkyl, n is a 1 to 4, x is the same as “n” described above, and y is the same as “m” described above.

The silicone may be a polysiloxane corresponding to the following formula:

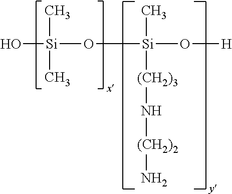

in which x′ and y′ are integers such that the weight-average molecular weight (Mw) is comprised between about 5000 and 500 000;
b) amino silicones corresponding to following formula:

R′ₐG₃₋ₐ-Si(OSiG₂)ₙ-(OSiG_{b}R′_{2-b})ₘ—O-SiG₃₋ₐ-R′ₐ

in which:
G, which may be identical or different, designate a hydrogen atom, or a phenyl, OH or C₁-C₈ alkyl group, for example methyl, or C₁-C₈ alkoxy, for example methoxy,
a, which may be identical or different, denote the number 0 or an integer from 1 to 3, in particular 0;
b denotes 0 or 1, and in particular 1;
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;
R′, which may be identical or different, denote a monovalent radical having formula —CqH₂qL in which q is a number ranging from 2 to 8 and L is an optionally quaternized amino group chosen from the following groups:
—NR″-Q-N(R″)₂
—N(R″)₂
—N+(R″)₃ A-
—N+H(R″)₂ A-
—N+H₂(R″) A-
—N(R″)-Q-N+R″H₂ A-
—NR″-Q-N+(R″)₂H A-
—NR″-Q-N+(R″)₃ A-,
   in which R″, which may be identical or different, denote hydrogen, phenyl, benzyl, or a saturated monovalent hydrocarbon-based radical, for example a C₁-C₂₀ alkyl radical; Q denotes a linear or branched CrH₂ᵣ group, r being an integer ranging from 2 to 6, preferably from 2 to 4; and A- represents a cosmetically acceptable ion, in particular a halide such as fluoride, chloride, bromide or iodide.

Another group of amino silicones corresponding to this definition is represented by silicones having the following formula:

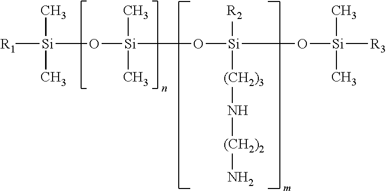

in which:
m and n are numbers such that the sum (n+m) can range from 1 to 1000, in particular from 50 to 250 and more particularly from 100 to 200, it being possible for n to denote a number from 0 to 999 and in particular from 49 to 249, and more particularly from 125 to 175, and for m to denote a number from 1 to 1000 and in particular from 1 to 10, and more particularly from 1 to 5;
R₁, R₂, R₃, which may be identical or different, represent a hydroxy or C₁-C₄ alkoxy radical, where at least one of the radicals R₁ to R₃ denotes an alkoxy radical.

The alkoxy radical is preferably a methoxy radical. The hydroxy/alkoxy mole ratio ranges preferably from 0.2:1 to 0.4:1 and preferably from 0.25:1 to 0.35:1 and more particularly equals 0.3:1. The weight-average molecular weight (Mw) of the silicone ranges preferably from 2,000 to 1,000,000, more particularly from 3,500 to 200,000.

Another group of amino silicones corresponding to this definition is represented by the following formula:

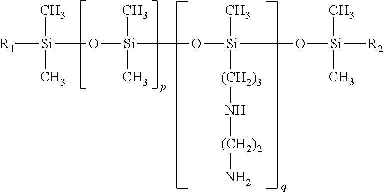

in which:
p and q are numbers such that the sum (p+q) ranges from 1 to 1000, particularly from 50 to 350, and more particularly from 150 to 250; it being possible for p to denote a number from 0 to 999 and in particular from 49 to 349, and more particularly from 159 to 239 and for q to denote a number from 1 to 1000, in particular from 1 to 10, and more particularly from 1 to 5;
R₁, R₂, which may be the same or different, represent a hydroxy or C₁-C₄ alkoxy radical, where at least one of the radicals R₁ or R₂ denotes an alkoxy radical.

The alkoxy radical is preferably a methoxy radical. The hydroxy/alkoxy mole ratio ranges generally from 1:0.8 to 1:1.1 and preferably from 1:0.9 to 1:1 and more particularly equals 1:0.95.

Another group of amino silicones is represented by the following formula:

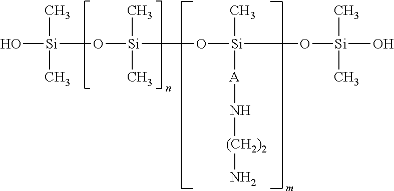

in which:
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;
A denotes a linear or branched alkylene radical containing from 4 to 8 carbon atoms and preferably 4 carbon atoms. This radical is preferably linear.

The weight-average molecular weight (Mw) of these amino silicones ranges preferably from 2000 to 1 000 000 and even more particularly from 3500 to 200 000.

Another group of amino silicones is represented by the following formula:

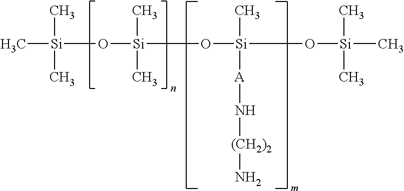

in which:
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;
A denotes a linear or branched alkylene radical containing from 4 to 8 carbon atoms and preferably 4 carbon atoms. This radical is preferably branched.

The weight-average molecular weight (Mw) of these amino silicones ranges preferably from 500 to 1 000 000 and even more particularly from 1000 to 200 000.

Another group of amino silicones is represented by the following formula:

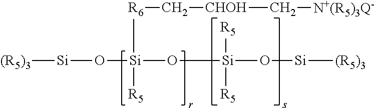

in which:
R₅ represents a monovalent hydrocarbon-based radical containing from 1 to 18 carbon atoms, and in particular a C₁-C₁ alkyl or C₂-C₁₅ alkenyl radical, for example methyl;
R₆ represents a divalent hydrocarbon-based radical, in particular a C₁-C₁₈ alkylene radical or a divalent C₁-C₁, for example C₁-C₈, alkylenoxy radical linked to the Si via an SiC bond;
Q- is an anion such as a halide ion, in particular chloride, or an organic acid salt (for example acetate);
r represents a mean statistical value from 2 to 20 and in particular from 2 to 8;
s represents a mean statistical value from 20 to 200 and in particular from 20 to 50.

Such amino silicones are described more particularly in U.S. Pat. No. 4,185,087.

A group of quaternary ammonium silicones is represented by the following formula:

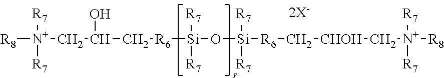

in which:
R₇, which may be identical or different, represent a monovalent hydrocarbon-based radical containing from 1 to 18 carbon atoms, and in particular a C₁-C₁₈ alkyl radical, a C₂-C₁₈ alkenyl radical or a ring containing 5 or 6 carbon atoms, for example methyl;
R₆ represents a divalent hydrocarbon-based radical, in particular a C₁-C₁₈ alkylene radical or a divalent C₁-C₁, for example C₁-C₈, alkylenoxy radical linked to the Si via an SiC bond;
R₈, which may be identical or different, represent a hydrogen atom, a monovalent hydrocarbon-based radical containing from 1 to 18 carbon atoms, and in particular a C₁-C₁₈ alkyl radical, a C₂-C₁₈ alkenyl radical or a —R₆—NHCOR₇ radical;
X— is an anion such as a halide ion, in particular chloride, or an organic acid salt (for example acetate);
r represents a mean statistical value from 2 to 200 and in particular from 5 to 100. These silicones are described, for example, in patent application EP-A 0530974.

A group of quaternary ammonium silicones is represented by the following formula:

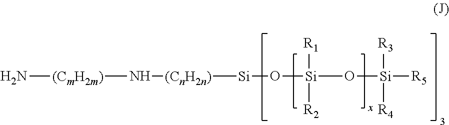

in which:
R₁, R₂, R₃ and R₄, which may be identical or different, denote a C₁-C₄ alkyl radical or a phenyl group;
R₅ denotes a C₁-C₄ alkyl radical or a hydroxyl group;
n is an integer ranging from 1 to 5;
m is an integer ranging from 1 to 5;
and in which x is chosen such that the amine number is between 0.01 and 1 meq/g;
multiblockpolyoxyalkylenated amino silicones, of type (AB)ₙ, A being a polysiloxane block and B being a polyoxyalkylenated block containing at least one amine group.

Said silicones are preferably constituted of repeating units having the following general formulae:

[—(SiMe₂O)xSiMe₂-R—N(R″)—R′—O(C₂H₄O)ₐ(C₃H₆₀)b—R′—N(H)—R—]

or alternatively

[—(SiMe₂O)xSiMe₂-R—N(R″)—R′—O(C₂H₄O)ₐ(C₃H₆₀)b-]

in which:
a is an integer greater than or equal to 1, preferably ranging from 5 to 200, more particularly ranging from 10 to 100;
b is an integer comprised between 0 and 200, preferably ranging from 4 to 100, more particularly between from 5 and 30;
x is an integer ranging from 1 to 10 000, more particularly from 10 to 5000;
R″ is a hydrogen atom or a methyl;
R, which may be identical or different, represent a divalent linear or branched C₂-C₁₂ hydrocarbon-based radical, optionally including one or more heteroatoms such as oxygen; preferably, R denotes an ethylene radical, a linear or branched propylene radical, a linear or branched butylene radical, or a —CH₂CH₂CH₂OCH(OH)CH₂—radical; preferentially R denotes a —CH₂CH₂CH₂OCH(OH)CH₂—radical;
R′, which may be identical or different, represent a divalent linear or branched C₂-C₁₂ hydrocarbon-based radical, optionally including one or more heteroatoms such as oxygen; preferably, R′ denotes an ethylene radical, a linear or branched propylene radical, a linear or branched butylene radical, or a —CH₂CH₂CH₂OCH(OH)CH₂—radical; preferentially R′ denotes —CH(CH₃)—CH₂—.

The siloxane blocks preferably represent between 50 and 95 mol % of the total weight of the silicone, more particularly from 70 to 85 mol %.

The amine content is preferably between 0.02 and 0.5 meq/g of copolymer in a 30% solution in dipropylene glycol, more particularly between 0.05 and 0.2. The weight-average molecular weight (Mw) of the silicone oil is preferably comprised between 5000 and 1,000,000, more particularly between 10,000 and 200,000.

Non-limiting examples of amino-functionalized silicones include bis-hydroxy/methoxy amodimethicones, bis-cetearyl amodimethicone, amodimethicone, bis(C₁₃₋₁₅ alkoxy) PG amodimethicones, aminopropyl phenyl trimethicones, aminopropyl dimethicones, bis-amino PEG/PPG-41/3 aminoethyl PG-propyl dimethicones, caprylyl methicones, and a mixture thereof. In some instances, a particularly useful amino-functionalized silicone is bis-hydroxy/methoxy amodimethicone, wherein X is isobutyl and one of the R is OH and the other is OCH₃ in the above structure, also known as “Bis-Hydroxy/Methoxy Amodimethicone” and “3-[(2-aminoethyl)amino]-2-methylpropyl Me, di-Me, [(hydroxydimethylsilyl)oxy]- and [(methoxydimethylsilyl)oxy]-terminated.” Bis-hydroxy/methoxy amodimethicone is commercially available under the tradename DOWSIL AP-8087 FLUID from The Dow Chemical Company. A particularly preferred amino-functionalized silicone is amodimethicone” A non-limiting example of amodimethicone products containing amino silicones having structure (D) re sold by Wacker under the name BELSIL ADM 652, BELSIL ADM 4000 E, or BELSIL ADM LOG 1. A product containing amino silicones having structure (E) is sold by Wacker under the name FLUID WR 1300. Additionally or alternative, the weight-average molecular weight (Mw) of the silicone ranges preferably from 2,000 to 200,000, even more particularly 5,000 to 100,000 and more particularly from 10,000 to 50,000.

In a preferred embodiment, the one or more amino-functionalized silicones are selected from amodimethicone, bis-hydroxy/methoxy amodimethicone, bis-cetearyl amodimethicone, bis(C13-15 alkoxy) PG amodimethicone, aminopropyl phenyl trimethicone, aminopropyl dimethicone, bis-amino PEG/PPG-41/3 aminoethyl PG-propyl dimethicone, or a mixture thereof. In a further preferred embodiments, the amino-functionalized silicone is amodimethicone.

The total amount of the one or more amino-functionalized silicones in the compositions will vary. Nonetheless, in various embodiments, the compositions include about 1 to about 18 wt. % of the one or more amino-functionalized silicones, based on the total weight of the composition. In further embodiments, the compositions include about 1 to about 15 wt. %, about 1 to 12 wt. %, about 1 to about 10 wt. %, about 1 to about 8 wt. %, about 1 to about 6 wt. %, about 2 to about 18 wt. %, about 2 to about 12 wt. %, about 2 to about 10 wt. %, about 2 to about 10 wt. %, about 2 to about 8 wt. %, about 2 to about 6 wt. %, about 3 to about 18 wt. %, about 3 to about 15 wt. %, about 3 to about 12 wt. %, about 3 to about 10 wt. %, about 3 to about 8 wt. %, about 3 to about 6 wt. %, about 4 to about 18 wt. %, about 4 to about 15 wt. %, about 4 to about 12 wt. %, about 4 to about 10 wt. %, about 4 to about 8 wt. %, about 4 to about 6 wt. %, about 1 wt. %, about 2 wt. %, about 3 wt. %, about 4 wt. %, about 5 wt. %, about 6 wt. %, about 7 wt. %, or about 8 wt. %, of the one or more amino-functionalized silicones based on the total weight of the composition.

### (g) Water-Soluble Solvents

The term “water soluble organic solvent” is interchangeable with the terms “water soluble solvent” and “water-miscible solvent” and means a compound that is liquid at 25° C. and at atmospheric pressure (760 mmHg), and it has a solubility of at least 50% in water under these conditions. In some cases, the water-soluble solvents has a solubility of at least 60%, 70%, 80%, or 90%. Non-limiting examples of water-soluble solvents include, for example, organic solvents selected from glycerin, alcohols (for example C₁₋₈, or C₁₋₄ alcohols), polyols (polyhydric alcohols), glycols, and a mixture thereof.

Nonlimiting examples of water-soluble organic solvents. Non-limiting examples of water-soluble organic solvents include, for example, organic solvents selected from glycerin, alcohols (for example, C₁₋₁₀, C₁₋₈, or C₁₋₄ alcohols), polyols (polyhydric alcohols), glycols, and a mixture thereof. Nonlimiting examples of monoalcohols and polyols include ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin.

Further non-limiting examples of water soluble organic solvents include alkanediols (polyhydric alcohols) such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

Polyhydric alcohols are useful. Examples of polyhydric alcohols include glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a mixture thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, and a mixture thereof.

In a preferred embodiment, the composition include one or more glycols selected from glycerin, propylene glycol, butylene glycol, pentylene glycol, hexylene glycol, caprylyl glycol, dipropylene glycol, and mixtures thereof.

The total amount of the one or more water-soluble solvents in the compositions, if present, will vary. Nonetheless, in various embodiments, the compositions include about 0.1 to about 20 wt. % of the one or more water soluble solvents, based on the total weight of the compositions. In further embodiments, the compositions include about 0.1 to about 15 wt. %, about 0.1 to about 10 wt. %, about 0.1 to about 8 wt. %, about 0.1 to about 5 wt. %, about 0.5 to about 20 wt. %, about 0.5 to about 15 wt. %, about 0.5 to about 10 wt. %, about 0.5 to about 8 wt. %, about 0.5 to about 5 wt. %, about 1 to about 20 wt. %, about 1 to about 15 wt. %, about 1 to about 10 wt. %, about 1 to about 8 wt. %, about 1 to about 5 wt. %, about 2 to about 20 wt. %, about 2 to about 15 wt. %, about 2 to about 10 wt. %, 2 to about 8 wt. %, about 2 to about 5 wt. %, about 1 wt. %, about 1.5 wt. %, about 2 wt. %, about 2.5 wt. %, about 3 wt. %, about 3.5 wt. %, about 4 wt. %, about 4.5 wt. %, or about 5 wt. % of the one or more water-soluble solvents, based on a total weight of the composition.

### (h) Water

The total amount of water in the hair treatment compositions will vary. Nonetheless, in various embodiments, the hair treatment compositions include about 50 to about 90 wt. % of water, based on the total weight of the compositions. In further embodiments, the hair treatment composition includes about 60 to about 90 wt. %, about 70 to about 90 wt. %, about 50 to about 85 wt. %, about 60 to about 85 wt. %, about 70 to about 85 wt. %, about 50 to about 80 wt. %, about 60 to about 80 wt. %, about 70 to about 80 wt. %, about 65 to about 85 wt. %, about 60 wt. %, about 65 wt. %, about 70 wt. %, about 75 wt. %, about 80 wt. %, or about 85 wt. %, based on the total weight of the compositions.

### (i) Non-Silicone-Based Fatty Compounds

The term “fatty substance” means an organic compound without silicone that is insoluble in water at ordinary temperature (25° C.) and at atmospheric pressure (760 mmHg), i.e. which has a solubility of less than 5%, preferably less than 1% and even more preferentially less than 0.1%. They have in their structure a hydrocarbon-based chain containing at least 6 carbon atoms.

More particularly, the one or more non-silicone-based fatty compounds may be selected from C₆-C₁₆ hydrocarbons, hydrocarbons containing more than 16 carbon atoms, non-silicone oils of animal origin, plant oils of triglyceride type, synthetic triglycerides, fluoro oils, fatty alcohols, non-salified fatty acids, fatty acid and/or fatty alcohol esters other than triglycerides and plant waxes, non-silicone waxes and silicones, and mixtures thereof.

Fatty alcohols, fatty esters and fatty acids more particularly contain one or more linear or branched, saturated or unsaturated hydrocarbon-based groups comprising 6 to 30 carbon atoms, which are optionally substituted, in particular, with one or more (in particular 1 to 4) hydroxyl groups. If they are unsaturated, these compounds may comprise one to three conjugated or unconjugated carbon-carbon double bonds.

As regards the C₆-C₁₆ hydrocarbons, they are linear, branched or optionally cyclic, and are preferably alkanes. Examples that may be mentioned include hexane, dodecane and isoparaffins such as isohexadecane and isodecane.

A hydrocarbon-based oil of animal origin that may be mentioned is perhydrosqualene.

The triglyceride oils of plant or synthetic origin are preferably chosen from liquid fatty acid triglycerides comprising from 6 to 30 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, castor oil, avocado oil, caprylic/capric acid triglycerides, for instance those sold by the company Stearineries Dubois or those sold under the names Miglyol® 810, 812 and 818 by the company Dynamit Nobel, jojoba oil and shea butter oil.

The linear or branched hydrocarbons of mineral or synthetic origin containing more than 16 carbon atoms are preferably chosen from liquid paraffins, petroleum jelly, liquid petroleum jelly, polydecenes and hydrogenated polyisobutene such as Parleam®.

The fluoro oils may be chosen from perfluoromethylcyclopentane and perfluoro-1,3-dimethylcyclohexane, sold under the names Flutec® PC1 and Flutec® PC3 by the company BNFL Fluorochemicals; perfluoro-1,2-dimethylcyclobutane; perfluoroalkanes such as dodecafluoropentane and tetradecafluorohexane, sold under the names PF 5050® and PF 5060® by the company 3M, or bromoperfluorooctyl sold under the name Foralkyl® by the company Atochem; nonafluoromethoxybutane and nonafluoroethoxyisobutane; perfluoromorpholine derivatives such as 4-trifluoromethyl perfluoromorpholine sold under the name PF 5052® by the company 3M.

The fatty alcohols that may be used in the cosmetic composition of step a) are saturated or unsaturated, linear or branched alcohols comprising from 6 to 30 carbon atoms and more particularly from 8 to 30 carbon atoms, among which mention may be made, for example, of cetyl alcohol, stearyl alcohol and the mixture thereof (cetylstearyl alcohol or cetearyl alcohol), octyldodecanol, 2-butyloctanol, 2-hexyldecanol, 2-undecylpentadecanol, oleyl alcohol or linoleyl alcohol.

The non-salified fatty acids that may be used in the cosmetic composition of step a) may be saturated or unsaturated carboxylic acids comprising from 6 to 30 carbon atoms and in particular from 9 to 30 carbon atoms. They are more particularly chosen from myristic acid, palmitic acid, stearic acid, behenic acid, oleic acid, linoleic acid, linolenic acid and isostearic acid.

These acids are not salified. This means that they are introduced in the form of free acids and that the composition does not comprise any alkaline agent leading to their salification.

The esters of fatty acids and/or of fatty alcohols, advantageously different from the triglycerides mentioned above, which may be used in the cosmetic composition used in step a) are esters of saturated or unsaturated, linear or branched C₁-C₂₆ aliphatic mono- or polyacids and of saturated or unsaturated, linear or branched C₁-C₂₆ aliphatic mono- or polyalcohols, the total carbon number of the esters more particularly being greater than or equal to 10. Among the monoesters, mention may be made of dihydroabietyl behenate; octyldodecyl behenate; isocetyl behenate; cetyl lactate; C₁₂-C₁₅ alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methylacetyl ricinoleate; myristyl stearate; octyl isononanoate; 2-ethylhexyl isononate; octyl palmitate; octyl pelargonate; octyl stearate; octyldodecyl erucate; oleyl erucate; ethyl and isopropyl palmitates, 2-ethylhexyl palmitate, 2-octyldecyl palmitate, alkyl myristates such as isopropyl, butyl, cetyl, 2-octyldodecyl, myristyl or stearyl myristate, hexyl stearate, butyl stearate, isobutyl stearate; dioctyl malate, hexyl laurate, 2-hexyldecyl laurate.

Still within the context of this variant, esters of C₄-C₂₂ dicarboxylic or tricarboxylic acids and of C₁-C₂₂ alcohols and esters of mono-, di- or tricarboxylic acids and of C₂-C₂₆ di-, tri-, tetra- or pentahydroxy alcohols may also be used.

Mention may be made in particular of: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate, tridecyl erucate; triisopropyl citrate; triisostearyl citrate; glyceryl trilactate; glyceryl trioctanoate; trioctyldodecyl citrate; trioleyl citrate, propylene glycol dioctanoate; neopentyl glycol diheptanoate; diethylene glycol diisononanoate; and polyethylene glycol distearates.

Among the esters mentioned above, it is preferred to use ethyl, isopropyl, myristyl, cetyl or stearyl palmitates, 2-ethylhexyl palmitate, 2-octyldecyl palmitate, alkyl myristates such as isopropyl, butyl, cetyl or 2-octyldodecyl myristate, hexyl stearate, butyl stearate, isobutyl stearate; dioctyl malate, hexyl laurate, 2-hexyldecyl laurate, isononyl isononanoate or cetyl octanoate.

The esters according to this variant may also be chosen from monoesters, diesters, triesters, tetraesters and polyesters, and mixtures thereof. These esters may be, for example, oleates, laurates, palmitates, myristates, behenates, cocoates, stearates, linoleates, linolenates, caprates and arachidonates, or mixtures thereof such as, especially, oleopalmitate, oleostearate and palmitostearate mixed esters. More particularly, use is made of monoesters and diesters and in particular mono- or di-oleate, -stearate, -behenate, -oleopalmitate, -linoleate, -linolenate or -oleostearate of sucrose, glucose or methylglucose.

The non-silicone wax(es) that may be used in the cosmetic composition used in step a) are chosen especially from carnauba wax, candelilla wax, esparto grass wax, hydrocarbon waxes including paraffin wax, ozokerite and microcrystalline wax, plant waxes such as olive wax, rice wax, hydrogenated jojoba wax or the absolute waxes of flowers such as the essential wax of blackcurrant blossom sold by the company Bertin (France), animal waxes, for instance beeswaxes or modified beeswaxes (cerabellina); other waxes or waxy starting materials that may be used according to the invention are especially marine waxes such as the product sold by the company Sophim under the reference M82, and polyethylene waxes or polyolefin waxes in general.

In a preferred embodiment, the one or more non-silicone-based fatty compounds are selected from oils, waxes, linear or branched alkanes, fatty esters, esters of fatty acids, esters of fatty alcohols, cetyl esters, triglycerides, or a mixture thereof.

The total amount of the one or more non-silicone-based fatty compounds in the compositions, if present, will vary. Nonetheless, in various embodiments, the compositions include about 0.1 to about 20 wt. % of the one or more non-silicone-based fatty compounds, based on the total weight of the compositions. In further embodiments, the compositions include about 0.1 to about 15 wt. %, about 0.1 to about 12 wt. %, about 0.1 to about 10 wt. %, about 0.1 to about 8 wt. %, about 0.1 to about 5 wt. %, about 0.5 to about 20 wt. %, about 0.5 to about 15 wt. %, about 0.5 to about 12 wt. %, about 0.5 to about 10 wt. %, about 0.5 to about 8 wt. %, about 0.5 to about 5 wt. %, about 1 to about 20 wt. %, about 1 to about 15 wt. %, about 1 to about 12 wt. %, about 1 to about 10 wt. %, about 1 to about 8 wt. %, about 1 to about 5 wt. %, about 2 to about 20 wt. %, about 2 to about 15 wt. %, about 2 to about 12 wt. %, about 2 to about 10 wt. %, about 2 to about 8 wt. %, about 2 to about 5 wt. %, or about 3 to about 5 wt. %, based on the total weight of the compositions.

### (j) Cationic Conditioning Polymer

Cationic polymers for purposes of the instant disclosure are polymers bearing a positive charge or incorporating cationic entities in their structure. The cationic polymers can comprise mixtures of monomer units derived from amine- and/or quaternary ammonium-substituted monomer and/or compatible spacer monomers. Cationic polymers often provide conditioning benefits to the hair treatment compositions and therefore may be referred to as “cationic conditioning polymers.” Non-limiting examples of cationic polymers include copolymers of 1-vinyl-2-pyrrolidine and 1-vinyl-3-methyl-imidazolium salt (e.g., chloride salt) (referred to as Polyquaternium-16); copolymers of 1-vinyl-2-pyrrolidine and dimethylaminoethyl methacrylate (referred to as Polyquaternium-11); cationic diallyl quaternary ammonium-containing polymer including, for example, dimethyldiallyammonium chloride homopolymer and copolymers of acrylamide and dimethyldiallyammonium chloride (referred to as Polyquaternium-6 and Polyquaternium-7); polysaccharide polymers, such as cationic cellulose derivatives and cationic starch derivatives. Cationic cellulose is available as salts of hydroxyethyl cellulose reacted with trimethyl ammonium substituted epoxide (referred to as Polyquaternium-10). Another type of cationic cellulose includes the polymeric quaternary ammonium salts of hydroxyethyl cellulose reacted with lauryl dimethyl ammonium-substituted epoxide (referred to as Polyquaternium-24). Additionally or alternatively, the cationic conditioning polymers may include or be chosen from cationic guar gum derivatives, such as guar hydroxypropyltrimonium chloride.

Preferred cationic polymers include cationic polysaccharide polymers, such as cationic cellulose, cationic starch, and cationic guar gum. In the context of the instant disclosure cationic polysaccharide polymers include cationic polysaccharides and polysaccharide derivatives (e.g., derivatized to be cationic), for example, resulting in cationic cellulose (cellulose derivatized to be cationic), cationic starch (derivatized to be cationic), cationic guar (guar derivatized to be cationic).

Non-limiting examples of cationic celluloses include hydroxyethylcellulose (also known as HEC), hydroxymethylcellulose, methylhydroxyethylcellulose, hydroxypropylcellulose (also known as HPC), hydroxybutylcellulose, hydroxyethylmethylcellulose (also known as methyl hydroxyethylcellulose) and hydroxypropylmethylcellulose (also known as HPMC), cetyl hydroxyethylcellulose, polyquaternium-10, polyquaternium-24, and mixtures thereof, preferably polyquaternium-10, polyquaternium-24, and mixtures thereof.

Non-limiting examples of cationic guar include guar hydroxypropyltrimonium chloride, hydroxypropyl guar hydroxypropyltrimonium chloride, guar hydroxypropyltrimethylammonium chloride, and mixtures thereof.

Non-limiting examples of cationic starch include starch hydroxypropyltrimonium chloride, hydroxypropyl oxidized starch PG trimonium chloride, and a mixture thereof.

In certain embodiments, the hair treatment composition may include one or more polyquaterniums. Nonlimiting examples include polyquaternium-1, polyquaternium-2, polyquaternium-3, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-21, polyquaternium-22, polyquaternium-23, polyquaternium-24, polyquaternium-25, polyquaternium-26, polyquaternium-27, polyquaternium-28, polyquaternium-29, polyquaternium-30, polyquaternium-40, polyquaternium-41, polyquaternium-42, polyquaternium-43, polyquaternium-44, polyquaternium-45, polyquaternium-46, polyquaternium-47, polyquaternium-48, polyquaternium-49, polyquaternium-50, polyquaternium-51, polyquaternium-52, polyquaternium-53, polyquaternium-54, polyquaternium-55, polyquaternium-56, polyquaternium-57, polyquaternium-58, polyquaternium-59, polyquaternium-60, polyquaternium-61, polyquaternium-62, polyquaternium-63, polyquaternium-64, polyquaternium-65, polyquaternium-66, polyquaternium-67, etc. In some cases, preferred polyquaternium compounds include polyquaternium-10, polyquaternium-11, polyquaternium-67, and a mixture thereof.

In certain embodiments, the hair treatment composition may include polyquaternium-1 (ethanol, 2,2′,2″-nitrilotris-, polymer with 1,4-dichloro-2-butene and N,N,N′,N′-tetramethyl-2-butene-1,4-diamine), polyquaternium-2, (poly[bis(2-chloroethyl) ether-alt-1,3-bis[3-(dimethylamino)propyl]urea]), polyquaternium-4, (hydroxyethyl cellulose dimethyl diallylammonium chloride copolymer; Diallyldimethylammonium chloride-hydroxyethyl cellulose copolymer), polyquaternium-5 (copolymer of acrylamide and quaternized dimethylammoniumethyl methacrylate), polyquaternium-6 (poly(diallyldimethylammonium chloride)), polyquaternium-7 (copolymer of acrylamide and diallyldimethylammonium chloride), polyquaternium-8 (copolymer of methyl and stearyl dimethylaminoethyl ester of methacrylic acid, quaternized with dimethylsulphate), polyquaternium-9 (homopolymer of N,N-(dimethylamino)ethyl ester of methacrylic acid, quaternized with bromomethane), polyquaternium-10 (quaternized hydroxyethyl cellulose), polyquaternium-11 (copolymer of vinylpyrrolidone and quaternized dimethylaminoethyl methacrylate), polyquaternium-12 (ethyl methacrylate/abietyl methacrylate/diethylaminoethyl methacrylate copolymer quaternized with dimethyl sulfate), polyquaternium-13 (ethyl methacrylate/oleyl methacrylate/diethylaminoethyl methacrylate copolymer quaternized with dimethyl sulfate), polyquaternium-14 (trimethylaminoethylmethacrylate homopolymer), polyquaternium-15 (acrylamide-dimethylaminoethyl methacrylate methyl chloride copolymer), Polyquaternium-16 (copolymer of vinylpyrrolidone and quaternized vinylimidazole), Polyquaternium-17 (adipic acid, dimethylaminopropylamine and dichloroethylether copolymer), Polyquaternium-18 (azelanic acid, dimethylaminopropylamine and dichloroethylether copolymer), polyquaternium-19 (copolymer of polyvinyl alcohol and 2,3-epoxypropylamine), polyquaternium-20 (copolymer of polyvinyl octadecyl ether and 2,3-epoxypropylamine), polyquaternium-22 (copolymer of acrylic acid and diallyldimethylammonium chloride), polyquaternium-24 (auaternary ammonium salt of hydroxyethyl cellulose reacted with a lauryl dimethyl ammonium substituted epoxide), polyquaternium-27 (block copolymer of Polyquaternium-2 and Polyquaternium-17), polyquaternium-28 (copolymer of vinylpyrrolidone and methacrylamidopropyl trimethylammonium), polyquaternium-29 (chitosan modified with propylen oxide and quaternized with epichlorhydrin), polyquaternium-30 (ethanaminium, N-(carboxymethyl)-N,N-dimethyl-2-[(2-methyl-1-oxo-2-propen-1-yl)oxy]-, inner salt, polymer with methyl 2-methyl-2-propenoate), polyquaternium-31 (N,N-dimethylaminopropyl-N-acrylamidine quatemized with diethylsulfate bound to a block of polyacrylonitrile), polyquaternium-32 (poly(acrylamide 2-methacryloxyethyltrimethyl ammonium chloride)), polyquaternium-33 (copolymer of trimethylaminoethylacrylate salt and acrylamide), polyquaternium-34 (copolymer of 1,3-dibromopropane and N,N-diethyl-N′,N′-dimethyl-1,3-propanediamine), Polyquaternium-35 (methosulphate of the copolymer of methacryloyloxyethyltrimethylammonium and of methacryloyloxyethyldimethylacetylammonium), polyquaternium-36 (copolymer of N,N-dimethylaminoethylmethacrylate and buthylmethacrylate, quaternized with dimethylsulphate), polyquaternium-37 (poly(2-methacryloxyethyltrimethylammonium chloride)), polyquaternium-39 (terpolymer of acrylic acid, acrylamide and diallyldimethylammonium Chloride), polyquaternium-42 (poly[oxyethylene(dimethylimino)ethylene (dimethylimino)ethylene dichloride]), Polyquaternium-43 (copolymer of acrylamide, acrylamidopropyltrimonium chloride, 2-amidopropylacrylamide sulfonate and dimethylaminopropylamine), polyquaternium-44 (3-Methyl-1-vinylimidazolium methyl sulfate-N-vinylpyrrolidone copolymer), polyquaternium-45 (copolymer of (N-methyl-N-ethoxyglycine)methacrylate and N,N-dimethylaminoethylmethacrylate, quaternized with dimethyl sulphate), polyquaternium-46 (terpolymer of vinylcaprolactam, vinylpyrrolidone, and quaternized vinylimidazole), polyquaternium-47 (terpolymer of acrylic acid, methacrylamidopropyl trimethylammonium chloride, and methyl acrylate), and/or polyquaternium-67.

In certain embodiments, the hair treatment compositions of the instant disclosure include one or more cationic polymers selected from cationic cellulose derivatives, quaternized hydroxyethyl cellulose (e.g., polyquaternium-10), cationic starch derivatives, cationic guar gum derivatives, copolymers of acrylamide and dimethyldiallyammonium chloride (e.g., polyquaternium-7), polyquaterniums, and a mixture thereof. For example, the cationic polymer(s) may be selected from polyquaterniums, for example, polyquaterniums selected from polyquaternium-4, polyquaternium-5, polyquaternium-6, polyquaternium-7, polyquaternium-10, polyquaternium-22, polyquaternium-37, polyquaternium-39, polyquaternium-47, polyquaternium-53, polyquaternium-67 and a mixture thereof. A combination of two or more polyquaterniums can be useful. A particularly preferred and useful cationic polymer is polyquaternium-10.

In certain embodiments, the hair treatment compositions include one or more cationic polymers chosen from cationic proteins and cationic protein hydrolysates (e.g., hydroxypropyltrimonium hydrolyzed wheat protein), quaternary diammonium polymers (e.g., hexadimethrine chloride), copolymers of acrylamide and dimethyldiallyammonium chloride, and mixtures thereof.

The hair treatment compositions according to the instant disclosure typically include about 0.1 to about 5 wt. % of one or more cationic conditioning polymers, based on the total weight of the hair treatment composition. The hair treatment compositions may include about 0.1 to about 4 wt. %, about 0.1 to about 3 wt. %, about 0.1 to about 2 wt. %, about 0.1 to about 1.5 wt. %, about 0.2 to about 5 wt. %, about 0.2 to about 4 wt. %, about 0.2 to about 3 wt. %, about 0.2 to about 2 wt. %, about 0.2 to about 1.5 wt. % of the one or more cationic polymers, based on the total weight of the hair treatment composition.

### (k) Miscellaneous Ingredients

The compositions optionally include or exclude (or are essentially free from) one or more miscellaneous ingredients. Miscellaneous ingredients are ingredients that are compatible with the compositions and do not disrupt or materially affect the basic and novel properties of the compositions. Nonlimiting examples of ingredients include preservatives, fragrances, pH adjusters, salts, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, etc. In various embodiments, the miscellaneous ingredients are chosen from preservatives, fragrances, pH adjusters, salts, chelating agents, buffers, composition colorants, and mixtures thereof. In the context of the instant disclosure, a “composition colorant” is a compound that colors the composition but does not have an appreciable coloring effect on hair. In other words, the composition colorant is included to provide a coloring to the composition for aesthetic appeal but is not intended to impart coloring properties to hair. Styling gels, for example, can be found in a variety of different colors (e.g., light blue, light pink, etc.) yet application of the styling gel to hair does not visibly change the color of the hair.

The total amount of the one or more miscellaneous ingredients in the compositions, if present, will vary. Nonetheless, in various embodiments, the compositions include about 0.1 to about 15 wt. % of the one or more miscellaneous ingredients, based on the total weight of the compositions. In further embodiments, the compositions include about 0.1 to about 12 wt. %, about 0.1 to about 10 wt. %, about 0.1 to about 5 wt. %, about 0.5 to about 15 wt. %, about 0.5 to about 12 wt. %, about 0.5 to about 10 wt. %, about 0.5 to about 8 wt. %, about 0.5 to about 5 wt. %, about 1 to about 15 wt. %, about 1 to about 12 wt. %, about 1 to about 10 wt. %, about 1 to about 8 wt. %, about 1 to about 5 wt. %, about 2 to about 15 wt. %, about 2 to about 12 wt. %, about 2 to about 10 wt. %, about 2 to about 8 wt. %, or about 2 to about 5 wt. %, based on the total weight of the compositions.

### Viscosity

Viscosity measurements of the hair styling compositions may be carried out using Brookfield [RV] viscometer. The hair styling compositions typically have a viscosity of about 500 cSt to about 5,000 cSt at room temperature (25° C.) (Torque 50%, RH6 spindle, ViscoQC 100 Rotational Viscometer). Nonetheless, in various embodiments, the compositions have a viscosity of about 500 cSt to about 4,000 cSt, about 500 cSt to about 3,000 cSt, about 500 cSt to about 2,500 cSt, about 500 cSt to about 2,000 cSt, about 1,000 cSt to about 5,000 cSt, about 1,000 cSt to about 4,000 cSt, about 1,000 cSt to about 3,000 cSt, about 1,000 cSt to about 2,500 cSt, or about 1,000 cSt to about 2,000 cSt at room temperature (25° C.) (Torque 50%, RH6 spindle, ViscoQC 100 Rotational Viscometer).

### Exclusions

All components that are positively set forth in the instant disclosure may be negatively excluded from the claims, e.g., a claimed composition may be “free,” “essentially free” (or “substantially free”) of one or more components that are positively set forth in the instant disclosure.

In various embodiments, the hair treatment composition is free or essentially free from anionic surfactants.

In various embodiments, the hair treatment composition is free or essentially free from polymers, copolymers, and crosspolymers formed with acrylate or methacrylate monomers, e.g, free or essentially free from polyacrylic acid and polyacrylate polymers and crosspolymers.

In various embodiments, the hair treatment composition is free or essentially free from N-alkyl-2-mercaptoacetamide. In further embodiments, the hair treatment composition is free or essentially free from all mercaptoacetamides.

In various embodiments, the hair treatment composition is free or essentially free from ethylene carbonate. In further embodiments, the hair treatment composition is free or essentially free from free from linear carbonates, e.g., dimethyl carbonate, diethyl carbonate, etc. In various embodiments, the composition is free or essentially free from cyclic lactones (e.g., valerolactone, caprolactone, pantolactone, meadowlactone, etc.), free or essentially free from heterocyclic molecules (e.g., 2-oxazolidinone, 2-imidazolidinone, etc.) free or essentially free from sulfones (dimethyl sulfone, 2,3,4,5-tetrahydrothiophene-1,1-dioxide), and/or free or essentially free from ureas (e.g., urea, ethylene urea, etc.). In yet further embodiments, the hair treatment composition is free or essentially free from carbonates.

In various embodiments, the hair treatment compositions are free or essentially free from polysaccharides other than the cyclodextrin and derivatives thereof.

In various embodiments, the hair treatment composition is free or essentially free from silicones other than the one or more amino-functionalized silicones. Similarly, in various embodiments, the composition is free or essentially free from silicones other than amodimethicone.

In further embodiments, the hair treatment composition is free or essentially free from monosaccharides and disaccharides. For example, the composition is free or essentially free from ribose, arabinose, glucose, fructose, xylose, sucrose, and/or methyl glucoside.

In various embodiments, the hair treatment composition is free or essentially free from formaldehyde, derivatives of formaldehyde, formalin, and compounds that produce formaldehyde upon heating.

In further embodiments, the hair treatment composition is free or essentially free from thioglycolic acid, thiolactic acid, or salts thereof.

### EMBODIMENTS

In various embodiments, the hair treatment composition comprises or consists of:
(a) about 1 to about 5 wt. %, preferably about 1.5 to about 4, more preferably about 2 to about 4 wt. % of citric acid, a salt thereof, or a mixture thereof;
(b) about 0.5 to about 5 wt. %, preferably about 1 to about 4, more preferably about 1 to about 3 wt. % of a cyclodextrin or derivative thereof, preferably a cyclodextrin or derivative of α-cyclodextrin, ß-cyclodextrin, γ-cyclodextrin, methyl derivatives of α-cyclodextrin, ß-cyclodextrin, or γ-cyclodextrin, hydroxypropyl derivatives of α-cyclodextrin, ß-cyclodextrin, or γ-cyclodextrin, or mixtures thereof, more preferably ß-cyclodextrin;
   wherein a combined total amount of the citric acid, salt thereof, of combination thereof of (a) and the cyclodextrin or derivative thereof of (b) is about 2 to about 12 wt. %, preferably about 2 to about 10 wt. %, more preferably about 3 to about 8 wt. %, and even more preferably about 3 to about 6 wt. %;
   wherein preferably the citric acid, salts thereof, or combination thereof of (a) and the cyclodextrin or derivatives thereof of (b) have been individually combined with each other to form a composition in which the cyclodextrin or derivative thereof of (b) is dissolved in the citric acid, salt thereof, or combination thereof of (a) before subsequently being combined with additional components of the hair-treatment composition;
(c) about 1 to about 10 wt. %, preferably about 1 to about 8 wt. %, more preferably about 2 to about 6 wt. % of one or more cationic surfactants, preferably one or more mono-alkyl trimonium halide compounds, for example, more mono-alkyl trimonium halide compounds selected from cetrimonium chloride, steartrimonium chloride, behentrimonium chloride, cocotrimonium chloride, cocamidopropyltrimonium chloride. Preferred are cetrimonium chloride, steartrimonium chloride and behentrimonium chloride, even more preferably wherein the one or more cationic surfactants includes behentrimonium chloride, optionally in combination with cetrimonium chloride;
(d) about 0.1 to about 10 wt. %, preferably about 0.5 to about 8, more preferably about 1 to about 5 of one or more nonionic surfactants or emulsifiers, preferably wherein the one or more nonionic surfactants or emulsifiers are selected from alkoxylated fatty alcohols, fatty acid esters of polyoxyethylene glycol, ethoxylated mono or diglycerides, sorbitan esters, ethoxylated sorbitan esters, fatty acid glycol esters, ethylene oxide, alkyl(ether)phosphates, alkylpolyglucosides, or a mixture thereof, more preferably one or more alkyl or polyalkyl ethers of poly(ethylene oxide) containing at least one C8-C30 alkyl radical, with a number of ethylene oxide (EO) units ranging from 3 to 200, in particular, selected from laureth-3, laureth-4, laureth-7, laureth-23, ceteth-5, ceteth-7, ceteth-15, ceteth-23, oleth-5, oleth-7, oleth-10, oleth-12, oleth-20, oleth-50, phytosterol 30 EO, steareth-6, steareth-20, steareth-21, steareth-40, steareth-100, beheneth 100, ceteareth-7, ceteareth-10, ceteareth-15, ceteareth-25, pareth-3, pareth-23, C12-15 pareth-3, C12-13 pareth-4, C12-13 pareth-23, trideceth-3, trideceth-4, trideceth-5, trideceth-6, trideceth-7 and trideceth-10, or mixtures thereof;
(e) about 1 to about 15 wt. %, preferably about 2 to about 12, more preferably about 4 to about 10 wt. % of one or more fatty alcohols, preferably one or more fatty alcohols selected from fatty alcohols have from 12 to 24 carbon atoms, more preferably one or more fatty alcohols selected from one or more solid fatty alcohols, for example, cetyl alcohol, stearyl alcohol, behenyl alcohol and mixtures thereof;
(f) about 2 to about 18 wt. %, preferably about 2 to about 12 wt. %, more preferably about 3 to about 8 wt. % of one or more amino-functionalized silicones, preferably one or more amino-functionalized silicones selected from amodimethicone, bis-hydroxy/methoxy amodimethicone, bis-cetearyl amodimethicone, bis(C13-15 alkoxy) PG amodimethicone, aminopropyl phenyl trimethicone, aminopropyl dimethicone, bis-amino PEG/PPG-41/3 aminoethyl PG-propyl dimethicone, or a mixture thereof, more preferably wherein the one or more amino-functionalized silicones includes amodimethicone or is amodimethicone;
(g) optionally, about 0.1 to about 14 wt. %, preferably about 1 to about 12 wt. %, more preferably about 2 to about 8 wt. % of one or more water-soluble solvents, preferably wherein the one or more water soluble solvents are selected from glycerin, C₁-C₆ mono-alcohols, polyols (polyhydric alcohols), glycols, or a mixture thereof, more preferably wherein the one or more water soluble solvents are selected from glycerin, glycols (e.g., ethylene glycol, propylene glycol, butylene glycol, pentylene glycol, caprylyl glycol, etc.), or a combination thereof;
(h) about 60 to about 85 wt. %, preferably about 65 to about 80 wt. %, more preferably about 70 to about 80 wt. % of water;
(i) optionally, about 0.1 to about 12 wt. %, preferably about 0.5 to about 10 wt. %, more preferably about 1 to about 6 wt. % of one or more non-silicone-based fatty compounds, preferably one or more non-silicone-based fatty compounds selected from oils, waxes, linear or branched alkanes, fatty esters, esters of fatty acids, esters of fatty alcohols, cetyl esters, triglycerides, or a mixture thereof, more preferably wherein the one or more non-silicone-based fatty compounds is selected from oil, cetyle esters, or a mixture thereof;
(j) optionally, about 0.01 to about 5 wt. %, preferably about 0.1 to about 4 wt. %, more preferably about 0.1 to about 3 wt. % of one or more cationic conditioning polymers, preferably one or more cationic conditioning polymers selected from cationic guar gum derivatives, such as cationic guar or cationic guar derivatives (e.g., guar hydroxypropyltrimonium chloride), cationic proteins or derivative thereof (hydroxypropyltrimonium hydrolyzed wheat protein), polyquaterniums, or a combination thereof;
   wherein all weight percentages are based on a total weight of the composition.

The pH of the composition can vary. Nonetheless, in various embodiments, a pH less than 7 (an acidic pH) is desirable. For example, the pH can be from about 3 to about 6.5, about 3 to about 6, about 3 to about 5.5, about 3 to about 5, about 3 to about 4.5, about 3.5 to about 6.5, about 3.5 to about 6, about 3.5 to about 5.5, about 3.5 to about 5, or about 3.5 to about 4.5.

The total combined amount of the citric acid, salts thereof, or combination of (a) and the cyclodextrin or derivatives of (b) will vary. Nonetheless, in various embodiments the total combined amount of the citric acid, salts thereof, or combination of (a) and the cyclodextrin and derivatives of (b) is about 1 to about 12 wt. %, based on a total weight of the hair treatment composition. In further embodiments, the total combined amount of the citric acid, salts thereof, or combination of (a) and the cyclodextrin and derivatives of (b) is about 1 to about 10 wt. %, about 1 to about 8 wt. %, about 1 to about 6 wt. %, about 1 to about 5 wt. %, about 2 to about 12 wt. %, about 2 to about 10 wt. %, about 2 to about 8 wt. %, about 2 to about 6 wt. %, about 2 to about 5 wt. %, about 3 to about 12 wt. %, about 3 to about 10 wt. %, about 3 to about 8 wt. %, about 3 to about 6 wt. %, about 3 to about 5 wt. %, or about 1 wt. %, 2 wt. %, 3 wt. %, 4 wt. %, 5 wt. %, 6 wt. %, 7 wt. %, or 8 wt. %, based on a total weight of the hair treatment composition.

The weight ratio of the citric acid, salts thereof, or combination of (a) to the cyclodextrin or derivatives of (b) will vary. Nonetheless, in various embodiments, the citric acid, salts thereof, or combination of (a) and the cyclodextrin or derivatives of (b) are in a weight ratio of about of about 8:1 to about 1:2 ((a):(b)). In further embodiments, the citric acid, salts thereof, or combination of (a) and the cyclodextrin or derivatives of (b) are in a weight ratio of 6:1 to about 1:2, about 5:2 to about 1:2, about 4:1 to about 1:2, about 3:1 to about 1:2, about 2:1 to about 1:2, about 8:1 to about 1:1, about 6:1 to about 1:1, about 5:1 to about 1:1, about 4:1 to about 1:1, about 3:1 to about 1:1, about 2:1 to about 1:1, about 1.2:1, about 1.3:1, about 1.4:1, about 1.5:1, about 1.6:1, or about 1.8:1 ((a):(b)).

The mole ratio of the citric acid, salts thereof, or combination of (a) to the cyclodextrin or derivatives of (b) will vary. Nonetheless, in various embodiments, the citric acid, salts thereof, or combination of (a) and the cyclodextrin or derivatives of (b) are in a mole ratio of about 20:1 to about 3:1. In further embodiments, the citric acid, salts thereof, or combination of (a) and the cyclodextrin or derivatives of (b) are in a mole ratio of about 18:1 to about 3:1, about 15:1 to about 3:1, about 20:1 to about 5:1, about 18:1 to about 5:1, about 15:1 to about 5:1, about 20:1 to about 8:1, about 18:1 to about 8:1, about 15:1 to about 8:1, about 20:1 to about 10:1, about 18:1 to about 10:1, about 15:1 to about 10:1, about 14:1, about 13:1, about 12:1, or about 11:1.

In various embodiments, the citric acid, salts thereof, or combination of (a) and the cyclodextrin of (b) are combined with one another before being added into the hair treatment compositions of the instant disclosure. For example, the cyclodextrin is preferably solubilized in the citric acid to form a solubilized combination of citric acid and cyclodextrin. The combination can be heated to facilitate or hasten the dissolution of the cyclodextrin. The solubility of cyclodextrin in water is not always ideal. Therefore, combining the cyclodextrin with the citric acid and dissolving the cyclodextrin in citric acid before adding combining the combination with other components of the hair treatment composition can be beneficial.

### Methods

As explained throughout the instant disclosure, the hair treatment compositions are particularly useful in methods for treating hair, preferably human hair, in particular human hair of the head. Treatment with the compositions improves fiber alignment, reduces frizz, and impart smoothness to the hair. The methods typically comprise applying a hair treatment composition of the instant disclosure to the hair. The hair is preferably wet or damp, but the composition can also be applied to dry hair. Application of the compositions to the hair will moisten the hair because the compositions are aqueous (contain a large portion of water).

The hair treatment composition is allowed to remain on the hair for a period of time before being rinsed from the hair. In various embodiments, the hair treatment composition is allowed to remain on the hair for 1 minute to about 60 minutes before being rinsed from the hair. In further embodiments, the hair treatment composition is allowed to remain on the hair for about 1 minute to about 45 minutes, about 1 minute to about 30 minutes, about 1 minute to about 15 minutes, about 5 minutes to about 60 minutes, about 5 minutes to about 45 minutes, about 5 minutes to about 30 minutes, about 5 minutes to about 15 minutes, about 5 minutes, about 10 minutes, about 15 minutes, about 20 minutes, about 25 minutes, about 30 minutes, about 40 minutes, about 45 minutes, or about 60 minutes, before being rinsed from the hair. Along these lines, the hair treatment composition is considered a “rinse out” or “rinse off” product.

After rinsing the hair, the hair can be dried, for example, with a blow drier. After the hair is dry, the hair can be treated with a thermal treatment (treated with heat). For example, the hair can be treated with a hot iron, in particular, a flat iron. Typically, the hot iron is passed over the hair at least once, at least twice, at least three times, or more. The hot iron is preferably at a temperature of about 150° C. to about 300° C., preferably 150° C. to about 250° C., more preferably about 180° C. to about 230° C.

The treated hair exhibits improved hair fiber alignment, reduced frizz, and and smoothness. The treated hair is also soft, shiny, conditioned, with a healthy appearance.

Implementation of the present disclosure is provided by way of the following examples. The examples serve to illustrate the technology without being limiting in nature.

### Example 1

(Inventive Composition)

| | | A |
|---|---|---|
| (a) | CITRIC ACID | 2.5 |
| (b) | CYCLODEXTRIN | 1.5 |
| Total (a) + (b) | | 4 |
| Ratio of (a):(b) | | 1.7 |
| (c) | BEHENTRIMONIUM CHLORIDE | 2.5 |
| | AND CETRIMONIUM CHLORIDE | |
| (d) | TRIDECETH-5, TRIDECETH-10, | 1.4 |
| | TRIDECETH-6, GLYCERYL | |
| | LINOLENATE, GLYCERYL | |
| | OLEATE, GLYCERYL LINOLEATE, | |
| | AND/OR SAFFLOWER | |
| | GLUCOSIDE | |
| (e) | CETEARYL ALCOHOL | 6 |
| (f) | AMODIMETHICONE | 4.8 |
| (g) | GLYCERIN, BUTYLENE GLYCOL, | 3.4 |
| | CAPRYLYL GLYCOL, AND/OR | |
| | ISOPROPYL ALCOHOL | |
| (i) | CETYL ESTERS | 1.5 |
| | HELIANTHUS ANNUUS | 0.01 |
| | (SUNFLOWER) SEED OIL | |
| (j) | HYDROXYPROPYLTRIMONIUM | 0.1 |
| | HYDROLYZED WHEAT PROTEIN | |
| (k) | MISCELLANEOUS* | ≤5 |
| | (Fillers, pH adjusters, preservatives, | |
| | botanical extract, fragrances, | |
| | buffers, stabilizers, vitamins, and/or | |
| | emollients, etc.) | |
| (h) | WATER | QS |

| | |
|---|---|
| pH | 3-4 |

*e.g., Styrax benzoin gum, boron nitride, xylose, acetic acid, benzoic acid, iris Florentina root extract, fragrance phenoxyethanol, sodium citrate, BHT, sodium hydroxide, tocopherol, etc.

(Comparative Compositions)

| | | B | | | C |
|---|---|---|---|---|---|
| (c) | BEHENTRIMONIUM CHLORIDE | 3 | (a) | CITRIC ACID | 2.5 |
| | AND CETRIMONIUM CHLORIDE | | | | |
| (d) | TRIDECETH-6 | 0.4 | (b) | CYCLODEXTRIN | 1.8 |
| (e) | CETEARYL ALCOHOL | 7 | Total (a) + (b) | | 4.3 |
| (f) | AMODIMETHICONE | 4.8 | Ratio of (a):(b) | | 1.6:1 |
| (g) | GLYCERIN, BUTYLENE | 0.7 | (c) | GLYCERIN | 1 |
| | GLYCOL, CAPRYLYL GLYCOL, | | | | |
| | AND/OR ISOPROPYL ALCOHOL | | | | |
| (f) | CETYL ESTERS AND | 2 | (e) | MISCELLANEOUS* | ≤5 |
| | | | | (Fillers, pH adjusters, | |
| | | | | preservatives, | |
| | | | | botanical extract, | |
| | | | | fragrances, buffers, | |
| | | | | stabilizers, vitamins, | |
| | | | | and/or emollients, | |
| | | | | etc.) | |
| | HELIANTHUS ANNUUS | 0.01 | (d) | WATER | QS |
| | (SUNFLOWER) SEED OIL AND | | | | |
| | SCLEROCARYA BIRREA SEED | | | | |
| | OIL | | | | |
| (g) | MISCELLANEOUS* | ≤5 | pH | | 3-4 |
| (h) | WATER | QS | | | |

*Fillers, pH adjusters, preservatives, botanical extract, fragrances, buffers, stabilizers, vitamins, and/or emollients, etc.

### Example 3

### (Comparative Testing)

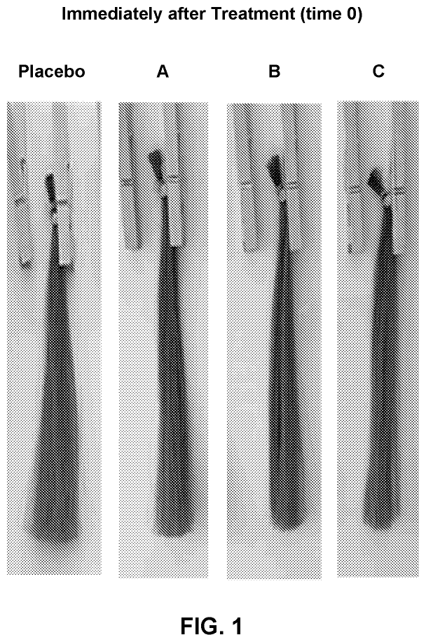

To determine how treatment with the Inventive Composition of Example 1 (Inventive Composition A) and with the comparative Compositions of Example 2 (Comparative Compositions B and C) influence hair, testing was carried out on commercially available hair swatches (CP3 bleached hair, 2 gm, 15 cm). All hair swatches were initially cleansed with a standard shampoo prior to treatment with one of compositions A-C. About 0.4 grams of each composition per gram of hair was applied to the cleansed hair swatches and distributed evenly throughout the hair. After distribution throughout the hair, the compositions were allowed to remain on the hair for 10 minutes, then thoroughly rinsed from the hair. The hair swatches were then blow dried and treated with a flat iron (180° C.). The flat iron was passed over the hair swatches three times for 10 seconds for each pass (the flat iron was passed along the distance of the hair swatches (15 cm) over a period of 10 seconds per pass). The swatches were evaluated, and pictures of the hair swatches are provided in FIG. 1.

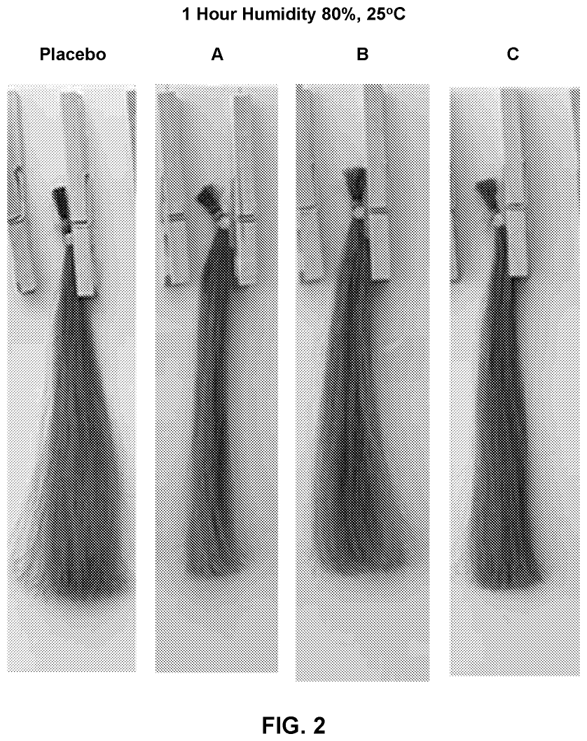

The hair swatches were then subjected to a humidity treatment. The hair swatches were placed in a humidity chamber having a temperature of 25° C. and 80% RH for 1 hour. After 1 hour in the humidity chamber, the swatches were again evaluated. Pictures of the hair swatches after humidity treatment are shown in FIG. 2.

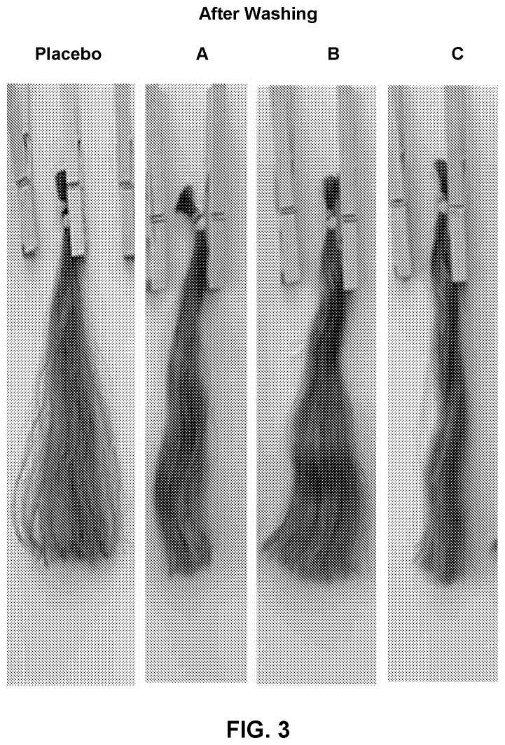

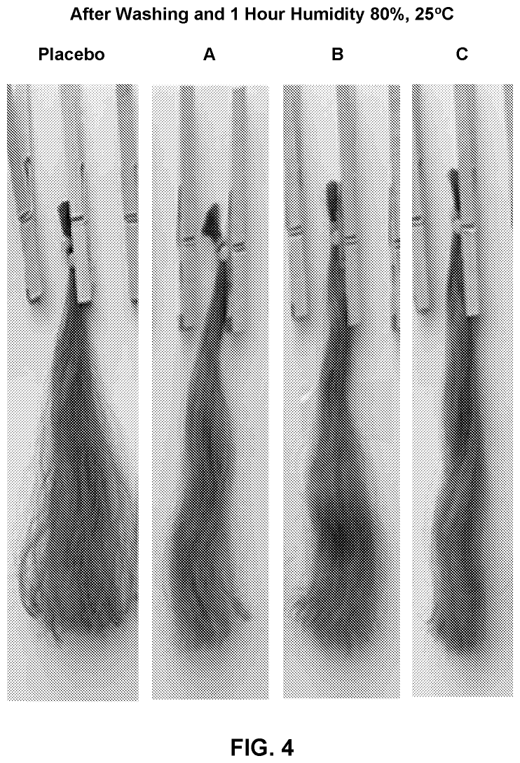

Following the humidity treatment, the swatches were again cleansed with the standard shampoo, blow dried and treated with a flat iron (180° C.) in the same manner as described above. The swatches were evaluated, and pictures of the swatches are provided in FIG. 3. The hair swatches were again subjected to the humidity treatment. The hair swatches were placed in the same humidity chamber having a temperature of 25° C. and 80% RH for 1 hour. After 1 hour in the humidity chamber, the swatches were again evaluated. Pictures of the swatches after treatment in the humidity chamber are shown in FIG. 4.

Hair swatches treated with Inventive Composition A consistently showed better fiber alignments, less frizz, more smoothness, and less roughness at the tips of the hair fibers, compared to the placebo and hair swatches treated with Comparative Compositions B and C.

The foregoing description illustrates and describes the disclosure. Additionally, the disclosure shows and describes only the preferred embodiments. However, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

As used herein, the terms “comprising,” “having,” and “including” are used in their open, non-limiting sense.

The terms “a,” “an,” and “the” are understood to encompass the plural as well as the singular. Thus, the term “a mixture thereof” also relates to “mixtures thereof.” Throughout the disclosure, the term “a mixture thereof” is used, following a list of elements as shown in the following example where letters A-F represent the elements: “one or more elements selected from the group consisting of A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counterion. This list of counterions, however, is non-limiting. Appropriate counterions for the components described herein are known in the art.

The expression “one or more” means “at least one” and thus includes individual components as well as mixtures/combinations.

The term “plurality” means “more than one” or “two or more.”

Other than in the operating examples, or where otherwise indicated, all numbers expressing quantities of ingredients and/or reaction conditions may be modified in all instances by the term “about,” meaning within +/−5% of the indicated number.

All percentages, parts and ratios herein are based upon the total weight of the compositions of the present invention, unless otherwise indicated.

Some of the various categories of components identified may overlap. In such cases where overlap may exist and the composition includes both components (or the composition includes more than two components that overlap), an overlapping compound does not represent more than one component. For example, certain compounds may be considered both a nonionic surfactant or emulsifier and a fatty compound. If a particular composition includes both a nonionic surfactant or emulsifier and a fatty compound, a single compound will serve as only the nonionic surfactant or emulsifier or only as the fatty compound (the single compound does not simultaneously serve as both the nonionic surfactant or emulsifier and the fatty component).

A “rinse-off” product refers to a composition that is rinsed and/or washed from the hair with water either after or during the application of the composition onto the hair, and before drying and/or styling the hair. At least a portion of the composition is removed from the hair during the rinsing and/or washing.

A “leave-on” product refers to a composition that is not rinsed and/or washed from the hair after or during application of the composition onto the hair. The composition remains on the hair during drying and/or throughout styling.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

The composition of the instant case optionally include one or more surfactants and/or emulsifiers, for example, one or more nonionic, anionic, cationic, and/or amphoteric/zwitterionic surfactants. The term “surfactants” and “emulsifiers” include salts of the surfactants and emulsifiers even if not explicitly stated. In other words, whenever the disclosure refers to a surfactant or emulsifier, it is intended that salts are also encompassed to the extent such salts exist, even though the specification may not specifically refer to a salt (or may not refer to a salt in every instance throughout the disclosure), for example, by using language such as “a salt thereof” or “salts thereof.” Sodium and potassium are common cations that form salts with surfactants and emulsifiers. However, additional cations such as ammonium ions, or alkanolammonium ions such as monoethanolammonium or triethanolammonium ions, may also form salts of surfactants.

The term “substantially free” or “essentially free” as used herein means that there is less than about 2% by weight of a specific material added to a composition, based on the total weight of the compositions. Nonetheless, the compositions may include less than about 1 wt. %, less than about 0.5 wt. %, less than about 0.1 wt. %, or none of the specified material.

All components that are positively set forth in the instant disclosure may be negatively excluded from the claims, e.g., a claimed composition may be “free,” “essentially free” (or “substantially free”) of one or more components that are positively set forth in the instant disclosure.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A hair treatment composition comprising:
(a) about 1 to about 8 wt. % of citric acid, a salt thereof, or a mixture thereof;
(b) about 0.5 to about 5 wt. % of a cyclodextrin or derivative thereof;
wherein a combined total amount of the citric acid of (a) and the cyclodextrin of (b) is about 2 to about 12 wt. %;
(c) about 1 to about 10 wt. % of one or more cationic surfactants;
(d) optionally, about 0.1 to about 5 wt. % of one or more nonionic surfactants or emulsifiers;
(e) about 1 to about 15 wt. % of one or more fatty alcohols;
(f) about 2 to about 18 wt. % of one or more amino-functionalized silicones;
(g) optionally, one or more water-soluble solvents;
(h) about 60 to about 85 wt. % of water;
wherein all weight percentages are based on a total weight of the composition.

2. The composition of claim 1 having a pH of about 3 to about 6.

3. The composition of claim 1, wherein (a) and (b) are in a weight ratio of about 4:1 to about 1:1 ((a):(b)) and/or a molar ratio of about 20:1 to about 5:1.

4. The composition of claim 1, wherein the citric acid of (a) and the cyclodextrin of (b) of the composition are individually combined with each other to form a mixture in which the cyclodextrin is dissolved in the citric acid; and subsequently the mixture is combined with additional components of the hair treatment composition to form the hair treatment composition.

5. The composition of claim 1, wherein the cyclodextrin or derivatives thereof are selected from α-cyclodextrin, ß-cyclodextrin, γ-cyclodextrin, methyl-α-cyclodextrin, methyl-ß-cyclodextrin, methyl-γ-cyclodextrin, or a mixture thereof.

6. The composition of claim 1, wherein the one or more cationic surfactants are selected from cetrimonium chloride, steartrimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, or a mixture thereof.

7. The composition of claim 1, wherein the one or more nonionic surfactants or emulsifiers are selected from alkoxylated fatty alcohols, fatty acid esters of polyoxyethylene glycol, ethoxylated mono or diglycerides, sorbitan esters, ethoxylated sorbitan esters, fatty acid glycol esters, ethylene oxide, alkyl(ether)phosphates, alkylpolyglucosides, or a mixture thereof.

8. The composition of claim 1, wherein the one or more fatty alcohols are selected from C14-22 linear alcohols.

9. The composition of claim 1, wherein the one or more amino-functionalized silicones are selected from amodimethicone, bis-hydroxy/methoxy amodimethicone, bis-cetearyl amodimethicone, bis(C13-15 alkoxy) PG amodimethicone, aminopropyl phenyl trimethicone, aminopropyl dimethicone, bis-amino PEG/PPG-41/3 aminoethyl PG-propyl dimethicone, or a mixture thereof.

10. The composition of claim 1, wherein the one or more amino-functionalized sililcones is amodimethicone.

11. The composition of claim 1 comprising about 1 to about 20 wt. % of the one or more water soluble solvents.

12. The composition of claim 11, wherein the one or more water soluble solvents are selected from glycerin, C₁-C₆ mono-alcohols, polyols (polyhydric alcohols), glycols, or a mixture thereof.

13. The composition of claim 1, further comprising:
(i) about 1 to about 10 wt. % of one or more non-silicone-based fatty compounds.

14. The composition of claim 13, wherein the one or more non-silicone-based fatty compounds are selected from oils, waxes, linear or branched alkanes, fatty esters, esters of fatty acids, esters of fatty alcohols, cetyl esters, triglycerides, or a mixture thereof.

15. The composition of claim 1, further comprising:
(j) about 0.01 to about 5 wt. % of one or more cationic conditioning polymers.

16. The composition of claim 15, wherein the one or more cationic conditioning polymers are selected from polyquaternium-4, polyquaternium-10, polyquaternium-24, guar hydroxypropyltrimonium chloride, guar hydroxypropyltrimonium chloride, cocodimonium hydroxypropyl hydrolyzed rice protein, stearyldimoniumhydroxypropyl hydrolyzed rice protein, hydroxypropyltrimoniumhydrolyzed silk, cocodimonium hydroxypropyl hydrolyzed soy protein, lauryldimonium hydroxypropyl hydrolyzed soy protein, hydroxypropyltrimonium hydrolyzed soy protein, hydroxypropyltrimonium hydrolyzed vegetable protein, stearyldimonium hydroxypropyl hydrolyzed vegetable protein, cocodimonium hydroxypropyl hydrolyzed wheat protein, hydroxypropyltrimonium hydrolyzed wheat protein, stearyldimonium hydroxypropyl hydrolyzed wheat protein, or a mixture thereof.

17. The composition of claim 1, further comprising:
(k) about 0.1 to about 10 wt. % of one or more miscellaneous ingredients.

18. A hair treatment composition comprising:
(a) about 1 to about 8 wt. % of citric acid, a salt thereof, or a combination thereof;
(b) about 0.5 to about 5 wt. % of a cyclodextrin or derivative thereof;
wherein a combined total amount of the citric acid of (a) and the cyclodextrin of (b) is about 2 to about 10 wt. %; and
the citric acid, salt thereof, or combination thereof of (a) and the cyclodextrin or derivatives thereof of (b) have been individually combined with each other to form a composition in which the cyclodextrin or derivative thereof of (b) is dissolved in the citric acid, salt thereof, or combination thereof of (a) before subsequently being combined with additional components of the hair-treatment composition;
(c) about 1 to about 10 wt. % of one or more cationic surfactants;
(d) about 0.1 to about 5 wt. % of one or more nonionic surfactants or emulsifiers;
(e) about 1 to about 10 wt. % of one or more fatty alcohols;
(f) about 2 to about 18 wt. % of amodimethicone;
(g) about 1 to about 15 wt. % of the one or more water soluble solvents selected from glycerin, C₁-C₆ mono-alcohols, polyols (polyhydric alcohols), glycols, or a mixture thereof;
(h) about 60 to about 85 wt. % of water;
(i) about 1 to about 10 wt. % of one or more non-silicone-based fatty compounds;
(j) optionally, about 0.01 to about 5 wt. % of one or more cationic conditioning polymers; and
(k) optionally, about 0.1 to about 10 wt. % of one or more miscellaneous ingredients;
wherein all weight percentages are based on a total weight of the composition.

19. A method for treating hair comprising applying the composition of claim 1 to the hair.

20. The method of claim 19, further comprising rinsing the composition from the hair, drying the hair, and treating the hair with a flat iron at a temperature of about 150° C. to about 280° C.
